(19) United States
(12) Patent Application Publication
(10) Pub. No.: US 2009/0062369 A1
(43) Pub. Date: Mar. 5, 2009

(54) USE OF SECRETORY PHOSPHOLIPASE A2 (SPLA2) INHIBITORS TO DECREASE SPLA2 LEVELS

(76) Inventors: Joaquim Trias, Milbrae, CA (US); Colin Hislop, Menlo Park, CA (US)

Correspondence Address: PERKINS COIE LLP, POST OFFICE BOX 1208, SEATTLE, WA, 98111-1208, US

(21) Appl. No.: 11/849,243

(22) Filed: Aug. 31, 2007

(51) Int. Cl. A61K 31/404; A61P 9/00; A61P 9/04; A61P 9/10; G01N 33/53

(52) U.S. Cl. 514/419; 435/7.9

(57) ABSTRACT

Administration of sPLA₂ inhibitors has been found to decrease sPLA₂ levels in human serum. Provided herein are methods of decreasing serum sPLA₂ levels in a subject in need thereof, as well as methods for accurately measuring sPLA₂ levels in a serum sample.

### BACKGROUND

Phospholipases A₂ (PLA₂) are a superfamily of enzymes that hydrolyze the ester bond at the sn-2 position of phosphoglycerides to release free fatty acid and lysophospholipids. The superfamily is divided into three groups. One of these groups, secretory phospholipase A₂ (sPLA₂), includes small enzymes of around 14 kDa that requires millimolar concentrations of Ca²⁺ to function. sPLA₂ catalyzes the release of arachidonic acid from phospholipids during the inflammatory response. This in turn stimulates the production of leukotrienes, prostacyclins, and other inflammation mediators.

sPLA₂ expression has been correlated to a variety of disease conditions. Various methods for treating these conditions using sPLA₂ inhibiting compounds have been developed or proposed. Each of these methods relies on the ability of sPLA₂ inhibitors to inhibit the enzyme. Rather than simply inhibiting sPLA₂, it would be therapeutically advantageous to develop methods for also decreasing sPLA₂ levels.

### SUMMARY

In certain embodiments, methods are provided for decreasing sPLA₂ levels in a subject by administering a therapeutically effective amount of one or more sPLA₂ inhibitors. In certain of these embodiments, the one or more sPLA₂ inhibitors comprise A-001 or a pharmaceutically acceptable prodrug, salt, or solvate of A-001. In certain of these embodiments, the one or more sPLA₂ inhibitors comprise A-002, a prodrug of A-001.

In certain embodiments, methods are provided for measuring sPLA₂ levels in a serum sample using an ELISA assay. In this assay, serum test samples and standard curve samples are applied to one or more wells of a plate pre-coated with a capture antibody that specifically binds sPLA₂. In certain embodiments, test samples and standard curve samples are diluted using a diluent comprising purified human serum from which sPLA₂ has been depleted. In certain embodiments, the diluent comprises 100% purified human serum. In other embodiments, the diluent comprises about 10% to 100% purified human serum, in other embodiments about 1% to 10% purified human serum, in other embodiments about 0.1% to about 1% purified human serum, and in other embodiments about 0.01% to about 0.1%. In certain embodiments, test samples and standard curve samples may be diluted in multiple diluents. For example, duplicate test samples may be diluted in 100% purified human serum and 10% purified human serum. In certain embodiments, the capture antibody on the plate may bind a specific sPLA₂ polypeptide, such as for example sPLA₂ type IIA. An acetylcholinesterase (AChE) conjugate is applied to each sample on the plate, wherein the conjugate specifically binds to sPLA₂ at an epitope distinct from that bound by the capture antibody. In certain embodiments, the AChE conjugate is an AChE:Fab′ conjugate. The plate incubated at about 15 to 30° C. for about 15 to 25 minutes, then the wells are washed one or more times and Ellman's reagent is added to each well. The plate incubated at about 15 to 30° C. for about 60 to 120 minutes, and the absorbance of each well is read at 400 to 420 nm. In certain embodiments, absorbance is measured at 405 nm. The concentration of sPLA₂ in a serum sample is determined using a standard curve that plots absorbance versus sPLA₂ concentration for one or more standard curve samples of known sPLA₂ concentration.

In certain embodiments, the use of one or more sPLA₂ inhibitors for preparation of a medicament for decreasing sPLA₂ levels in a subject in need thereof are provided.

In addition to the exemplary embodiments described above, further embodiments and aspects will become apparent by reference to the drawings and by study of the following descriptions.

### BRIEF DESCRIPTION OF FIGURES

FIG. 1: sPLA₂ standard curves. sPLA₂ stock was diluted in purified neat serum, normal neat serum, or buffer only and subjected to an ELISA with a 30 minute development time. Curves were generated by plotting absorbance at 405 nm versus sPLA₂ concentration.

FIG. 2: sPLA₂ standard curves. sPLA₂ stock was diluted in purified neat serum, normal neat serum, or buffer only and subjected to an ELISA with a 60 minute development time. Curves were generated by plotting absorbance at 405 nm versus sPLA₂ concentration.

FIG. 3: sPLA₂ standard curves. sPLA₂ stock was diluted in purified neat serum, normal neat serum, or buffer only and subjected to an ELISA with a 90 minute development time. Curves were generated by plotting absorbance at 405 nm versus sPLA₂ concentration.

FIG. 4: sPLA₂ standard curves. sPLA₂ stock was diluted in purified neat serum, normal neat serum, or buffer only and subjected to an ELISA with a 120 minute development time. Curves were generated by plotting absorbance at 405 nm versus sPLA₂ concentration.

FIG. 5: sPLA₂ standard curves. sPLA₂ stock was diluted in normal serum, 10% purified serum, 1% purified serum, or 0.1% purified serum and subjected to an ELISA with a 60 minute development time. Curves were generated by plotting absorbance at 405 nm versus sPLA₂ concentration.

FIG. 6: sPLA₂ standard curves. sPLA₂ stock was diluted in normal serum, 10% purified serum, 1% purified serum, or 0.1% purified serum and subjected to an ELISA with a 90 minute development time. Curves were generated by plotting absorbance at 405 nm versus sPLA₂ concentration.

FIG. 7: sPLA₂ standard curve. sPLA₂ stock was diluted in purified serum and subjected to an ELISA with a 60 minute development time. The standard curve was generated by plotting absorbance at 405 nm versus sPLA₂ concentration.

FIG. 8: sPLA₂ standard curve. sPLA₂ stock was diluted in purified serum and subjected to an ELISA with a 90 minute development time. The standard curve was generated by plotting absorbance at 405 nm versus sPLA₂ concentration.

FIG. 9: sPLA₂ standard curve. sPLA₂ stock was diluted in purified serum and subjected to an ELISA with a 60 minute development time. The standard curve was generated by plotting absorbance at 405 nm versus sPLA₂ concentration.

FIG. 10: sPLA₂ standard curve. sPLA₂ stock was diluted in 10% purified serum and subjected to an ELISA with a 60 minute development time. The standard curve was generated by plotting absorbance at 405 nm versus sPLA₂ concentration.

FIG. 11: sPLA₂ standard curve. sPLA₂ stock was diluted in 1% purified serum and subjected to an ELISA with a 60 minute development time. The standard curve was generated by plotting absorbance at 405 nm versus sPLA₂ concentration.

FIG. 12: sPLA₂ standard curve. sPLA₂ stock was diluted in 0.1% purified serum and subjected to an ELISA with a 60 minute development time. The standard curve was generated by plotting absorbance at 405 nm versus sPLA₂ concentration.

FIG. 13: Overlay of standard curves from FIGS. 9-12. sPLA₂ stock was diluted in purified serum, 10% purified serum, 1% purified serum, or 0.1% purified serum and subjected to an ELISA with a 60 minute development time. Standard curves were generated by plotting absorbance at 405 nm versus sPLA₂ concentration.

FIG. 14: sPLA₂ standard curve. sPLA₂ stock was diluted in purified serum and subjected to an ELISA with a 90 minute development time. The standard curve was generated by plotting absorbance at 405 nm versus sPLA₂ concentration.

FIG. 15: sPLA₂ standard curve. sPLA₂ stock was diluted in 10% purified serum and subjected to an ELISA with a 90 minute development time. The standard curve was generated by plotting absorbance at 405 nm versus sPLA₂ concentration.

FIG. 16: sPLA₂ standard curve. sPLA₂ stock was diluted in 1% purified serum and subjected to an ELISA with a 90 minute development time. The standard curve was generated by plotting absorbance at 405 nm versus sPLA₂ concentration.

FIG. 17: sPLA₂ standard curve. sPLA₂ stock was diluted in 0.1% purified serum and subjected to an ELISA with a 90 minute development time. The standard curve was generated by plotting absorbance at 405 nm versus sPLA₂ concentration.

FIG. 18: Overlay of standard curves from FIGS. 14-17. sPLA₂ stock was diluted in purified serum, 10% purified serum, 1% purified serum, or 0.1% purified serum and subjected to an ELISA with a 90 minute development time. Standard curves were generated by plotting absorbance at 405 nm versus sPLA₂ concentration.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Abbreviations

AChE, acetylcholinesterase; CAD, coronary artery disease; CHD, coronary heart disease; CV, coefficient of variance; CVD, cardiovascular disease; EIA, enzyme immunometric assay; HDL, high density lipoprotein; HMG-CoA, hydroxymethyl glutaryl coenzyme A; hs-CRP, highly sensitive C-reactive protein; ICAM-1, intercellular adhesion molecule 1; IDL, intermediate density lipoprotein; IL, interleukin (e.g., IL-6, IL-8); MCP-1, monocyte chemotactic protein-1; MI, heart attack; MCP-1α, macrophage inflammatory protein 1 alpha; QC, quality control; SD, standard deviation; sPLA₂, secretory phospholipase A₂, TIA, transient ischemic attack; TNFα, tumor necrosis factor alpha; VCAM-1, vascular cell adhesion molecule 1; VLDL, very low density lipoprotein.

Existing commercial kits for measuring sPLA₂ in a biological fluid sample have not been validated for use with serum samples. Serum contains basal levels of sPLA₂, which makes it difficult to generate an accurate standard curve for calculating sPLA₂ levels below about 5 ng/ml in a sample (Boekholdt 2005). Provided herein is a novel method for accurately measuring sPLA₂ levels in serum using an EIA double-antibody sandwich technique. This method utilizes serum from which sPLA₂ has been depleted as a diluent.

The assay disclosed herein was developed using materials from a commercially available sPLA₂ (human type IIA) EIA (enzyme immunoassay) kit (Cayman Chemical Co., Ann Arbor, Mich., Catalog No. 585000) (“the Cayman kit”). In certain embodiments, the assay may be carried out using materials from the Cayman kit (e.g., buffers, plate, etc.). In other embodiments, the assay may be carried out using equivalent materials, such as for example materials from a different kit. Such equivalent materials will be readily apparent to one of ordinary skill in the art.

The method disclosed herein for measuring sPLA₂ levels in serum utilizes depleted serum as a diluent for both standard curve and test samples. “Depleted serum” or “purified serum” as used herein refers to serum from which substantially all endogenous sPLA₂ or all endogenous sPLA₂ of a particular type has been removed. In certain embodiments, “depleted serum” or “purified serum” refers to serum from which substantially all endogenous sPLA₂ type IIA has been removed. One method for removing endogenous sPLA₂ from serum is set forth in Example 1, below. However, other methods known in the art for depleting a specific protein from a fluid sample may be utilized.

Examples 2-4, below, detail the development and validation of the assay disclosed herein. The tested sPLA₂ range of the assay was 0.06 ng/ml 4 ng/ml. In certain embodiments, the assay is capable of detecting levels of sPLA₂ as low as 0.05 ng/ml in serum, and in certain embodiments less than 0.05 ng/ml.

The protocol for preparing standard curve, test, and quality control samples for use in the assay disclosed herein is set forth in Example 5. Samples are prepared according to this protocol, then loaded in duplicate onto a plate (Cayman catalog no. 485002 or equivalent) that has been pre-coated with a monoclonal sPLA₂ antibody (“capture antibody”) that specifically binds sPLA₂. In certain embodiments, the capture antibody may interact with a specific form of sPLA₂, such as for example sPLA₂ type IIA.

sPLA₂ acetylcholinesterase:Fab′ conjugate (AChE:Fab′) (“conjugate”) (Cayman catalog no. 485000 or equivalent) is reconstituted in EIA buffer (Cayman catalog no. 400060 or equivalent). Cayman EIA buffer is 1M phosphate, pH 7.0, 1% BSA, 4M NaCl, 10 mM EDTA, and 0.1% sodium azide. Reconstituted conjugate is added to each well of the plate. The conjugate binds a different sPLA₂ epitope than the capture antibody, allowing the capture antibody and the conjugate to form a “sandwich” that is immobilized on the plate. Excess reagents are washed away, and the plate is covered and incubated overnight at 2-8° C.

Prior to development, the wells are washed five to six times with wash buffer (Cayman catalog no. 400062 or equivalent). To develop the plate, Ellman's Reagent (Cayman catalog no. 400050 or equivalent) is reconstituted in water and added to each well according to manufacturer instructions. The reconstituted Ellman's Reagent must be used the same day it is prepared because it is unstable. Ellman's Reagent contains an AChE substrate. Specifically, Ellman's Reagent contains acetylthiocholine and 5,5′-dithio-bis-(2-nitrobenzoic acid). Hydrolysis of acetylthiocholine by acetylcholinesterase produces thiocholine, which reacts with 5,5′-dithio-bis-(2-nitrobenzoic acid) to produce 5-thio-2-nitrobenzoic acid. 5-thio-2-nitrobenzoic acid has a strong absorbance at 412 nm. The intensity of absorbance at this wavelength is directly proportional to the concentration of bound conjugate, which is in turn is directly proportional to the concentration of sPLA₂ present (Absorbance a [AChE:Fab′ conjugate] α [sPLA₂]). During the development step, the plate is shaken, preferably in the dark. In certain embodiments the development time for the assay is 0.5 to 3 hours, preferably 1 to 1.5 hours. At the end of the development period, the high sPLA₂ standards should have greater yellow color development than the zero standards. Absorbance of each well is measured at 400-420 nm using a micro-well plate reader. In certain embodiments, absorbance is measured at about 405 nm.

Absorbance readings for duplicate wells are averaged, and the relative standard deviation and % CV are calculated. Standard curve data are used to generate a four parameter curve, and quality control and test samples are measured against the appropriate standard curve (i.e., the standard curve with the same serum content). In order for a test to be considered valid, variation between duplicate wells should be no more than about 25%, and negative controls (blanks) for each standard curve should have a mean absorbance of less than about 0.3.

In one embodiment, three quality control samples are utilized: high (2 ng/ml), medium (1 ng/ml), and low (0.5 ng/ml). In this embodiment, the calculated concentration for each quality control sample should preferably be within the following value ranges:

| | Quality control sample values (ng/ml) | | | |
|---|---|---|---|---|
| Quality | Purified | | | |
| control | serum | 10% serum | 1% serum | 0.1% serum |
| sample | diluent | diluent | diluent | diluent |
| High | 1.17 to 2.43 | 1.38 to 2.87 | 1.26 to 2.63 | 1.40 to 2.92 |
| (2 ng/ml) | | | | |
| Medium | 0.55 to 1.15 | 0.70 to 1.45 | 0.65 to 1.34 | 0.68 to 1.41 |
| (1 ng/ml) | | | | |
| Low | 0.33 to 0.68 | 0.38 to 0.78 | 0.36 to 0.74 | 0.35 to 0.73 |
| (0.5 ng/ml) | | | | |

The method disclosed herein for measuring sPLA₂ levels in serum was utilized to determine the effect of administering certain sPLA₂ inhibitors on sPLA₂ levels in 84 human subjects. The subjects included 24 subjects with diabetes, 32 subjects with metabolic syndrome, and 75 subjects receiving statin treatment. Subjects were administered the sPLA₂ inhibitor A-002, a prodrug form of A-001, twice a day at varying dosages from 50 to 500 mg. The structures of A-002and A-001 are set forth below. At two weeks and four weeks after the first administration, sPLA₂ levels were measured and compared to pre-administration baseline levels. A-002 administration resulted in a substantial decrease in sPLA₂ levels across all subject subpopulations (i.e., diabetic/non-diabetic, metabolic syndrome/non-metabolic syndrome, and statin/non-statin) at all dosages tested in a non-dose-dependent manner.

The use of sPLA₂ inhibitors to treat certain conditions associated with sPLA₂ by inhibiting the sPLA₂ enzyme has been disclosed previously. However, the results disclosed herein provide the first example of an sPLA₂ inhibitor decreasing sPLA₂ levels rather than simply inhibiting enzyme activity. Therefore, in certain embodiments, a method is provided for decreasing sPLA₂ levels in a subject in need thereof by administering one or more sPLA₂ inhibitors. The term “subject” as used herein refers to any mammal, preferably a human. A “subject in need thereof” as used herein refers to a subject with a condition that may be treated by decreasing sPLA₂ levels, a subject who has previously had a condition that may be treated by decreasing sPLA₂ levels, or a subject deemed at risk for developing a condition that may be treated by decreasing sPLA₂ levels. In certain embodiments, a subject in need thereof may exhibit elevated sPLA₂ levels, may have exhibited elevated sPLA₂ levels in the past, or may have been deemed at risk for developing elevated sPLA₂ levels. In other embodiments, a subject in need thereof may exhibit sPLA₂ levels falling within a normal range, but may nonetheless have a condition that may be treated by further decreasing sPLA₂ levels.

In certain embodiments, a “subject in need thereof” as used herein refers to a subject having a condition associated with inflammation, or a subject who has had such a condition in the past or been deemed at risk for developing such a condition in the future. In certain embodiments, “a subject in need thereof” may exhibit elevated levels of one or more additional markers associated with inflammation, including but not limited to hs-CRP, IL-6, MCP-1, TNFα, IL-8, ICAM-1, VCAM-1, and MIP-1α. “Conditions associated with inflammation” as used herein include, for example, atherosclerosis and certain cardiovascular diseases, as well as other inflammatory conditions such as for example multiple sclerosis (Cunningham 2006), Alzheimer's disease (Moses 2006), sickle cell (Styles 1996), rheumatoid arthritis, and osteoarthritis (Jamal 1998). In certain embodiments, decreasing sPLA₂ levels in a subject results in treatment of a condition associated with inflammation. Therefore, in certain embodiments, methods are further provided for treating such conditions.

In certain embodiments, a “subject in need thereof” refers to a subject diagnosed with cardiovascular disease (CVD) or exhibiting one or more conditions associated with CVD, a subject who has been diagnosed with or exhibited one or more conditions associated with CVD in the past, or a subject who has been deemed at risk of developing CVD or one or more conditions associated with CVD in the future due to hereditary or environmental factors. “Cardiovascular disease” or “CVD” as used herein includes, for example, atherosclerosis, coronary artery disease (CAD), coronary heart disease (CHD), conditions associated with CAD and CHD, cervebrovascular disease and conditions associated with cerebrovascular disease, peripheral vascular disease and conditions associated with peripheral vascular disease, aneurysm, vasculitis, venous thrombosis, diabetes mellitus, and metabolic syndrome. “Conditions associated with CAD and CHD” as used herein include, for example, angina and myocardial infarction (MI; heart attack). “Conditions associated with cerebrovascular disease” as used herein include, for example, transient ischemic attack (TIA) and stroke. “Conditions associated with peripheral vascular disease” as used herein include, for example, claudication. “Conditions associated with CVD” as used herein include, for example, hypertension, hypercholesterolemia (elevated cholesterol levels), elevated triglyceride levels, elevated glucose levels, and low HDL/LDL levels. Therefore, in certain embodiments, a subject in need thereof may be a subject exhibiting elevated cholesterol levels, particularly elevated levels of LDL, VLDL, IDL, and/or total cholesterol, or a subject that has exhibited such elevated cholesterol levels in the past or has been deemed at risk for developing such elevated cholesterol levels. In certain embodiments, decreasing sPLA₂ levels in a subject results in treatment of CVD or one or more conditions associated with CVD. Therefore, in certain embodiments, methods are further provided for treating such conditions.

The terms “treat,” “treating,” or “treatment” as used herein with regards to a condition that may be treated by decreasing sPLA₂ levels refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. For example, with regard to atherosclerosis, “treatment” may refer to a decrease in the likelihood of developing atherosclerotic plaque deposits, a decrease in the rate of development of deposits, a decrease in the number or size of existing deposits, or improved plaque stability.

Metabolic syndrome is a disorder characterized by a group of metabolic risk factors. These factors include, for example, abdominal obesity, atherogenic dyslipidemia, elevated blood pressure, insulin resistance or glucose intolerance, prothrombotic state, and proinflammatory state. Subjects are generally classified as having metabolic syndrome if they meet three of the five following criteria: 1) abdominal obesity (waist circumference >35 inches in women, >40 inches in men); 2) low HDL levels (<50 mg/dL in women, <40 mg/dL in men); 3) high blood pressure (>130/85 mm Hg) or current treatment with antihypertensive medication; 4) high triglyceride levels (>150 mg/dL); and 5) impaired fasting glucose (blood glucose levels of 110-126 mg/dL). Subjects with metabolic syndrome are at increased risk of developing CAD, CHD, conditions associated with CAD and CHD, and type 2 diabetes.

CHD and CAD are the most common types of CVD. CHD and CAD occur when coronary arteries that supply blood to the heart become hardened and narrowed due to atherosclerosis. Elevated sPLA₂ levels have been implicated in the development of atherosclerosis and other types of CVD (Hurt-Camejo 2001; Boekholdt 2005).

In certain embodiments, sPLA₂ inhibitors for use in decreasing sPLA₂ levels may be administered in conjunction with one or more additional therapeutic compounds for treating a condition associated with elevated sPLA₂ levels. For example, sPLA₂ inhibitors may be administered with one or more compounds for treating CVD, such as for example one or more ACE inhibitors or nitrosated ACE inhibitors, angiotensin 11 receptor antagonists or nitrosated angiotensin II receptor antagonists, beta-adrenergic blockers or nitrosated beta-adrenergic blockers, calcium channel blockers, or antithrombotics such as aspirin or nitrosated aspirin. Likewise, sPLA₂ inhibitors may be administered in conjunction with one or more compounds for lowering blood cholesterol levels, such as for example one or more statins, bile acid sequestrants such as cholestyramine resin or colestipol hydrochloride, fibrates such as bezafibrate, clofibrate, fenofibrate, or gemfibrozil, niacin or niacin derivatives such as xanthinol niacinate, or other miscellaneous compounds such as dextrothyroxine. As disclosed herein, administration of sPLA₂ inhibitors was effective at decreasing sPLA₂ levels in subjects that had received or were receiving statins. Therefore, in certain preferred embodiments, sPLA₂ inhibitors are administered in conjunction with one or more statins. As used herein, “statin” refers to any compound that competitively inhibits HMG-CoA reductase, an enzyme that catalyzes the conversion of HMG-CoA to mevalonate. Examples of statins that may be used in conjunction with the compositions and methods disclosed herein include, but are not limited to, atorvastatin (marketed as Lipitor® or Torvast®; see, e.g., U.S. Pat. Nos. 4,681,893 or 5,273,995), cerivastatin (marketed as Lipobay®), fluvastatin (marketed as Lesco®; U.S. Pat. No. 4,739,073), lovastatin (marketed as Mevacor® or Altocor®; see, e.g., U.S. Pat. No. 4,231,938), mevastatin, pitavastatin (marketed as Livalo® or Pitava®), pravastatin (marketed as Pravachol®, Selektine®, or Lipostat®; see, e.g., U.S. Pat. No. 4,346,227), rosuvastatin (marketed as Crestor®), simvastatin (marketed as Zocor® or Lipex®; see, e.g., U.S. Pat. No. 4,444,784), and ezetimibe plus simvastatin (marketed as Vytorin®), as well as various pharmaceutically acceptable salts, stereoisomers, prodrugs, or nitroderivatives thereof. In some cases, such as for example with simvastatin, the active form of the statin is a metabolite formed in the body of a subject following administration. In other cases, statins are administered in their active form.

In certain embodiments, reduction of sPLA₂ levels in a subject may result in corresponding decrease in cholesterol levels. Therefore, in certain embodiments, methods are provided for decreasing cholesterol levels in a subject by administering a therapeutically effective amount of one or more sPLA₂ inhibitors. In these embodiments, one or more sPLA₂ inhibitors may be administered to a subject that exhibits elevated cholesterol levels, particularly elevated LDL levels, a subject who has previously exhibited elevated cholesterol levels, or a subject who is deemed at risk for developing high cholesterol levels due to hereditary or environmental factors. A decrease in cholesterol levels refers to a decrease in total cholesterol, a decrease in LDL only, or a decrease in one or more of LDL, VLDL, and/or IDL. In addition to decreasing cholesterol levels, decreasing sPLA₂ levels in a subject may result in an improved HDL/LDL ratio. This improvement may arise from a decrease in LDL levels, an increase in HDL levels, or some combination thereof.

In certain embodiments, reduction of sPLA₂ levels in a subject may result in corresponding decrease in inflammation. Therefore, in certain embodiments, methods are provided for reducing inflammation in a subject in need thereof by decreasing sPLA₂ levels. In certain of these embodiments, reduction in sPLA₂ levels may result in a corresponding decrease in blood levels of one or more markers associated with inflammation other than sPLA₂, such as for example hs-CRP, IL-6, MCP-1, TNFα, IL-8, ICAM-1, VCAM-1, and MIP-1α. In certain embodiments, reduction of inflammatory markers following administration of one or more sPLA₂ inhibitors is accompanied by a reduction in cholesterol levels. In other embodiments, a reduction in inflammatory marker levels is observed in the absence of a decrease in cholesterol levels.

A “therapeutically effective amount” as used herein is an amount of a composition that produces a desired therapeutic effect in a subject, such as preventing or treating a target condition or alleviating symptoms associated with the condition. In certain embodiments, a therapeutically effective amount of an sPLA₂ inhibitor may refer to an amount that results in a measurable decrease in serum sPLA₂ levels in a subject. In certain of these embodiments, a therapeutically effective amount of an sPLA₂ inhibitor results in a statistically significant decrease in serum sPLA₂ levels in a subject. In certain embodiments, sPLA₂ inhibitors may be administered from once or more times per day to once every month or once every several months. In certain preferred embodiments, sPLA₂ inhibitors may be administered once a day, and in other preferred embodiments sPLA₂ inhibitors may be administered twice a day. In certain embodiments, a therapeutically effective amount of an sPLA₂ inhibitor may be from about 5 to about 1,000 mg/dose, and in certain of these embodiments may be from about 50 to about 500 mg/dose. One of ordinary skill in the art will recognize that a therapeutically effective amount of a composition may vary depending upon a variety of factors, including but not limited to the characteristics of the therapeutic composition (including, e.g., activity, pharmacokinetics, pharmacodynamics, and bioavailability), the physiological condition of the subject (including, e.g., age, sex, disease type and stage, general physical condition, responsiveness to a given dosage, and type of medication), the nature of the pharmaceutically acceptable carrier or carriers in the composition, and the route of administration. One skilled in the clinical and pharmacological arts will be able to determine a therapeutically effective amount through routine experimentation, namely by monitoring a subject's response to administration of a composition and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005, the entire disclosure of which is incorporated by reference herein.

sPLA₂ inhibitors for use in the methods disclosed herein may be administered on a one-time basis or in multiple administrations. In those embodiments wherein sPLA₂ inhibitors are given in multiple administrations, they may be administered at set intervals over a particular time period determined in advance, or they may be administered indefinitely or until a particular therapeutic benchmark is reached, such as for example until a subject exhibits a serum sPLA₂ level below a specified threshold.

In certain embodiments, sPLA₂ inhibitors for use in the methods disclosed herein may be part of a composition comprising one or more additional therapeutic compounds and/or one or more pharmaceutically acceptable carriers, or may be administered in conjunction with one or more additional therapeutic compounds and/or pharmaceutically effective carriers. A “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, encapsulating material, stabilizing agent, or some combination thereof. Each component of the carrier must be “pharmaceutically acceptable” in that it must be compatible with the other ingredients of the composition. It also must be suitable for contact with any tissue, organ, or portion of the body that it may encounter, meaning that it must not carry a risk of toxicity, irritation, allergic response, immunogenecity, or any other complication that excessively outweighs its therapeutic benefits. Examples of pharmaceutically acceptable carriers for use in the present invention include, but are not limited to, microcrystalline cellulose, hydroxypropyl methylcellulose (HPMC), magnesium stearate, lactose, povidone, antioxidant agents such as butylated hydroxyanisole (BHA), 2,6,-di-tert-butyl-4-methylphenol (BHT), propyl gallate, ascorbic acid (either free acid or salt forms thereof), citric acid, edetate disodium, calcium metabisulfate, croscarmellose sodium (CCNa), citric acid, lactic acid, malic acid, succinic acid, tartaric acid, and ethylenediaminetetraacetic acid (EDTA).

sPLA₂ inhibitors for use in the methods disclosed herein may be administered by any administration pathway known in the art, including but not limited to oral, aerosol, enteral, nasal, ophthalmic, parenteral, or transdermal (e.g., topical cream or ointment, patch). “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. In certain embodiments, sPLA₂ inhibitors may be formed into oral dosage units, such as for example tablets, pills, or capsules. In certain embodiments, sPLA₂ inhibitors may be administered via a time release delivery vehicle, such as for example a time release capsule. A “time release delivery vehicle” as used herein refers to any delivery vehicle that releases active agent (i.e., sPLA₂ inhibitor) over a period of time rather than immediately upon administration. In other embodiments, sPLA₂ inhibitors may be administered via an immediate release delivery vehicle.

As used herein, an “sPLA₂ inhibitor” refers to any compound that inhibits the activity of sPLA₂. In certain embodiments, an sPLA₂ inhibitor for use in the compositions and methods disclosed herein may be an indole-based sPLA₂ inhibitor, meaning that the compound contains an indole nucleus having the structure:

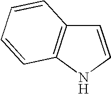

A variety of indole-based sPLA₂ inhibitors are known in the art. For example, indole-based sPLA₂ inhibitors that may be used in conjunction with the present invention include but are not limited to those set forth in U.S. Pat. No. 5,654,326 (Bach); U.S. Pat. No. 5,733,923 (Bach); U.S. Pat. No. 5,919,810 (Bach); U.S. Pat. No. 5,919,943 (Bach); U.S. Pat. No. 6,175,021 (Bach); U.S. Pat. No. 6,177,440 (Bach); U.S. Pat. No. 6,274,578 (Denney); and U.S. Pat. No. 6,433,001 (Bach), the entire disclosures of which are incorporated by reference herein. Methods of making indole-based sPLA₂ inhibitors are set forth in, for example, U.S. Pat. No. 5,986,106 (Khau); U.S. Pat. No. 6,265,591 (Anderson); and U.S. Pat. No. 6,380,397 (Anderson), the entire disclosures of which are incorporated by reference herein. sPLA₂ inhibitors for use in the present invention may be generated using these synthesis methods, or using any other synthesis method known in the art. In certain embodiments, sPLA₂ inhibitors for use in the present invention may be sPLA₂ type IIA, type V, and/or type X inhibitors.

In certain embodiments, sPLA₂ inhibitors for use in the current invention are 1H-indole-3-glyoxylamide compounds having the structure:

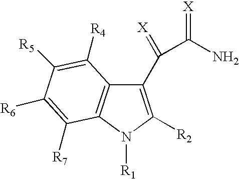

wherein:
each X is independently oxygen or sulfur;
R₁ is selected from the group consisting of (a), (b), and (c), wherein:

(a) is C₇-C₂₀ alkyl, C₇-C₂₀ alkenyl, C₇-C₂₀ alkynyl, carbocyclic radicals, or heterocyclic radicals;

(b) is a member of (a) substituted with one or more independently selected non-interfering substituents; and

(c) is the group-(L)-R₈₀, where, -(L)-is a divalent linking group of 1 to 12 atoms selected from carbon, hydrogen, oxygen, nitrogen, and sulfur, wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) carbon, hydrogen, and oxygen only; and where R₈₀ is a group selected from (a) or (b); R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;
R₄ and R₅ are independently selected from the group consisting of hydrogen, a non-interfering substituent, and -(Lₐ)-(acidic group), wherein -(Lₐ)- is an acid linker having an acid linker length of 1 to 4; provided that at least one of R₄ and R₅ must be -(Lₐ)-(acidic group);
R₆ and R₇ are each independently selected from hydrogen, non-interfering substituents, carbocyclic radicals, carbocyclic radicals substituted with non-interfering substituents, heterocyclic radicals, and heterocyclic radicals substituted with non-interfering substituents;
provided that for any of the groups R₁, R₆, and R₇, the carbocyclic radical is selected from the group consisting of cycloalkyl, cycloalkenyl, phenyl, naphthyl, norbornanyl, bicycloheptadienyl, tolulyl, xylenyl, indenyl, stilbenyl, terphenylyl, diphenylethylenyl, phenyl-cyclohexenly, acenaphthylenyl, and anthracenyl, biphenyl, bibenzylyl and related bibenzylyl homologues represented by the formula (bb),

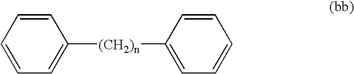

where n is a number from 1 to 8; provided, that for any of the groups R₁, R₆, and R₇, the heterocyclic radical is selected from the group consisting of pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo(1.2-A)pyridinyl, benzotriazolyl, anthranilyl, 1,2-benzisoxazolyl, benzoxazolyl, benzotriazolyl, purinyl, pryidinyl, dipyridylyl, phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl; and provided that for the groups R₁, R₂, R₄, R₅, R₆, and R₇ the non-interfering substituent is selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙCO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl, where n is from 1 to 8; and pharmaceutically acceptable salts, solvates, prodrug derivatives, racemates, tautomers, or optical isomers thereof.

In certain of these embodiments, -(L)- has the formula:

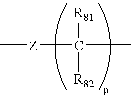

wherein R₈₁ and R₈₂ are each independently selected from the group consisting of hydrogen, C₁-C₁₀ alkyl, carboxy, carbalkoxy, and halo; p is a number from 1 to 5; and Z is selected from the group consisting of a bond, —(CH₂)—, —O—, —N(C₁-C₁₀ alkyl)-, —NH—, and —S—.

In certain of these embodiments wherein R₄ is -(Lₐ)-(acidic group), the acid linker -(Lₐ)- has the formula:

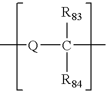

wherein Q is selected from the group consisting of —(CH₂)—, —O—, —NH—, and —S—; and R₈₃ and R₈₄ are each independently selected from the group consisting of hydrogen, C₁-C₁₀ alkyl, aryl, C₁-C₁₀ alkaryl, C₁-C₁₀ aralkyl, hydroxy, and halo.

In certain of these embodiments wherein R₅ is -(Lₐ)-(acidic group), the acid linker -(Lₐ)- has the formula:

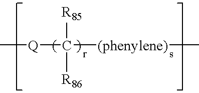

wherein r is a number from 2 to 7; s is 0 or 1; Q is selected from the group consisting of —(CH₂)—, —O—, —NH—, and —S—; and R₈₅ and R₈₆ are each independently selected from the group consisting of hydrogen, C₁-C₁₀ alkyl, aryl, C₁-C₁₀ alkaryl, C₁-C₁₀ aralkyl, carboxy, carbalkoxy, and halo.

In certain embodiments, a 1H-indole-3-glyoxylamide compound for use in the present invention is selected from the group consisting of: ((3-(2-Amino-1,2-dioxoethyl)-2-ethyl-1-(phenylmethyl)-1H-indol-4-yl)oxy)acetic acid; [[3-(2-Amino-1,2-dioxoethyl)-2-ethyl-1-(phenylmethyl)-1H-indol-4-yl]oxy]acetic acid methyl ester; ((3-(2-Amino-1,2-dioxoethyl)-2-methyl-1-(phenylmethyl)-1H-indol-4-yl)oxy)acetic acid; dl-2-((3-(2-Amino-1,2-dioxoethyl)-2-methyl-1-(phenylmethyl)-1H-indol-4-yl) oxy)propanoic acid; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-(1,1′-biphenyl)-4-ylmethyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid; ((3-(2-Amino-1,2-dioxoethyl)-2-cyclopropyl-1-(phenylmethyl)-1H-indol-4-yl)oxy)acetic acid; ((3-(2-Amino-1,2-dioxoethyl)-1-((1,1′-biphenyl)-2-ylmethyl)-2-cyclopropyl-1H-indol-4-yl)oxy)acetic acid; and 4-((3-(2-Amino-1,2-dioxoethyl)-2-ethyl-1-(phenylmethyl)-1H-indol-5-yl)oxy)butanoic acid, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

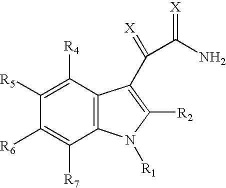

wherein:
both X are oxygen;
R₁ is selected from the group consisting of:

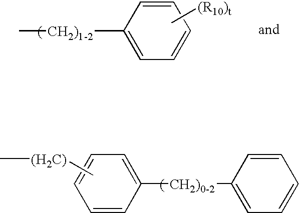

wherein R₁₀ is a radical independently selected from halo, C₁-C₁₀ alkoxy, —S—(C₁-C₁₀ alkyl), and C₁-C₁₀ haloalkyl, and t is a number from 0 to 5; R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

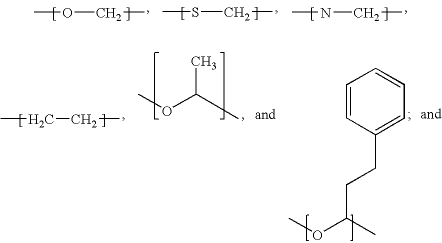

provided that the acid linker -(Lₐ)- for R₅ is selected from the group consisting of:

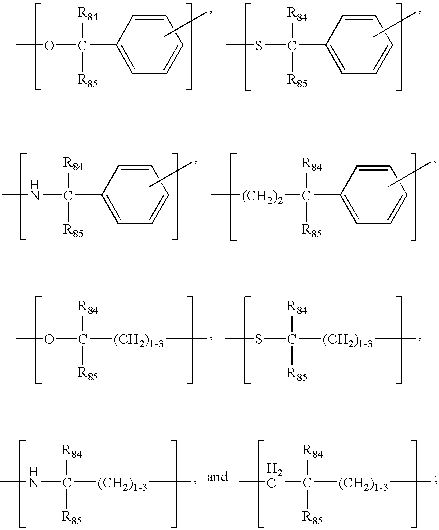

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)- (acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, 1H-indole-3-glyoxylamide compounds for use in the present invention are selected from the group consisting of: ((3-(2-Amino-1,2-dioxoethyl)-2-methyl-1-(phenylmethyl)-1H-indol-4-yl)oxy)acetic acid; ((3-(2-Amino-1,2-dioxoethyl)-2-methyl-1-(phenylmethyl)-1H-indol-4-yl)oxy)acetic acid methyl ester; dl-2-((3-(2-Amino-1,2-dioxoethyl)-2-methyl-1-(phenylmethyl)-1H-indol-4-yl) oxy)propanoic acid; dl-2-((3-(2-Amino-1,2-dioxoethyl)-2-methyl-1-(phenylmethyl)-1H-indol-4-yl) oxy)propanoic acid methyl ester; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-2-ylmethyl)-2-methyl-1H-indol-4-yl)oxy)acetic acid methyl ester; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid methyl ester; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid methyl ester; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid methyl ester; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid methyl ester; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid methyl ester; ((3-(2-Amino-1,2-dioxoethyl)-1-((3-chlorophenyl)methyl)-2-ethyl-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid methyl ester; ((3-(2-Amino-1,2-dioxoethyl)-1-((1,1′-biphenyl)-2-ylmethyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid methyl ester; ((3-(2-amino-1,2-dioxoethyl)-1-((1,1′-biphenyl)-2-ylmethyl)-2-propyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid methyl ester; ((3-(2-Amino-1,2-dioxoethyl)-2-cyclopropyl-1-(phenylmethyl)-1H-indol-4-yl) oxy)acetic acid; ((3-(2-Amino-1,2-dioxoethyl)-2-cyclopropyl-1-(phenylmethyl)-1H-indol-4-yl)oxy)acetic acid methyl ester; ((3-(2-Amino-1,2-dioxoethyl)-1-((1,1′-biphenyl)-2-ylmethyl)-2-cyclopropyl-1H-indol-4-yl)oxy)acetic acid; ((3-(2-Amino-1,2-dioxoethyl)-1-((1,1′-biphenyl)-2-ylmethyl)-2-cyclopropyl-1H-indol-4-yl)oxy)acetic acid methyl ester; 4-((3-(2-Amino-1,2-dioxoethyl)-2-ethyl-1-(phenylmethyl)-1H-indol-5-yl)oxy)butanoic acid; 4-((3-(2-Amino-1,2-dioxoethyl)-2-ethyl-1-(phenylmethyl)-1H-indol-5-yl)oxy)butanoic acid tert-butyl ester, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

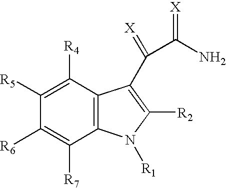

wherein:
each X is independently oxygen or sulfur;
R₁ is selected from groups (a), (b), and (c) wherein:

(a) is C₇-C₂₀ alkyl, C₇-C₂₀ alkenyl, C₇-C₂₀ alkynyl, carbocyclic radical, or heterocyclic radical;

(b) is a member of (a) substituted with one or more independently selected non-interfering substituents; and

(c) is the group -(L)-R₈₀, wherein -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) and carbon, hydrogen, and oxygen only; and where R₈₀ is a group selected from (a) or (b); R₂ is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), and a non-interfering substituent having a total of 1 to 3 atoms other than hydrogen; R₄ and R₅ are independently selected from the group consisting of hydrogen, a non-interfering substituent, and the group -(Lₐ)-(acidic group), wherein -(Lₐ)- is an acid linker having an acid linker length of 1 to 4; provided that at least one of R₄ and R₅ is -(Lₐ)-(acidic group);
R₆ and R₇ are each independently selected from the group consisting of hydrogen, non-interfering substituents, carbocyclic radicals, carbocyclic radicals substituted with non-interfering substituents, heterocyclic radicals, and heterocyclic radicals substituted with non-interfering substituents;
and pharmaceutically acceptable salts, solvates, prodrug derivatives, racemates, tautomers, or optical isomers thereof.

In certain embodiments, sPLA₂ inhibitors for use in the current invention are methyl ester prodrug derivatives of 1H-indole-3-glyoxylamide compounds having the structure:

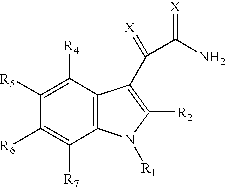

wherein:
both X are oxygen;
R₁ is selected from the group consisting of:

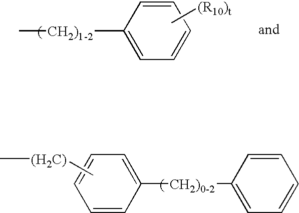

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

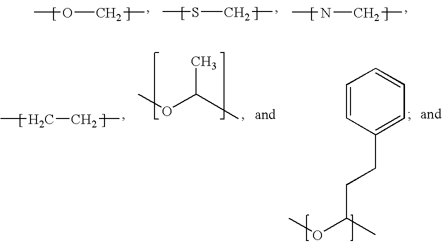

provided that the acid linker -(Lₐ)- for R₅ is selected from the group consisting of:

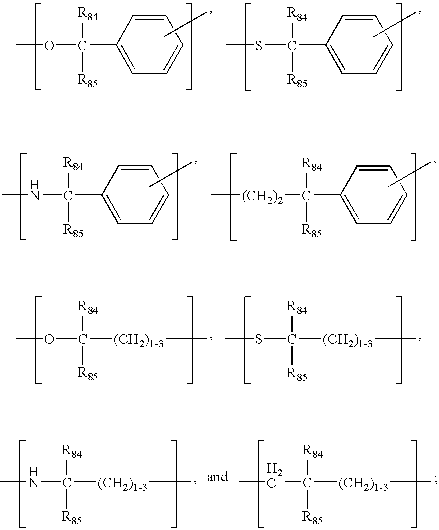

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)- (acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are (acyloxy) alkyl ester prodrug derivatives of 1H-indole-3-glyoxylamide compounds having the structure:

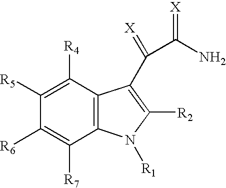

wherein:
both X are oxygen;
R₁ is selected from the group consisting of:

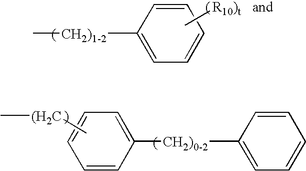

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

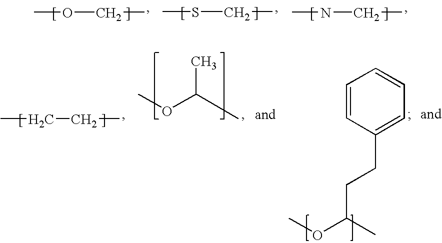

provided that the acid linker -(Lₐ)- for R₅ is selected from the group consisting of:

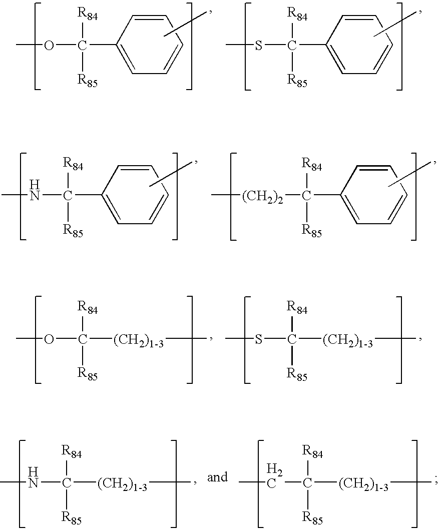

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

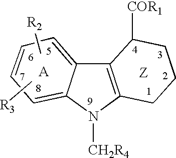

wherein:
R₁ is selected from the group consisting of —NHNH₂ and —NH₂;
R₂ is selected from the group consisting of —OH and —O(CH₂)ₘR₅; wherein R₅ is selected from the group consisting of H, —CO₂H, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

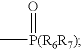

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ is selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; wherein R₀ and R₁₀ are each independently —(C₁-C₄)alkyl or -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, and phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy, or naphthyl;
A is selected from the group consisting of phenyl and pyridyl wherein the nitrogen is at the 5-, 6-, 7-, or 8-position;
Z is selected from the group consisting of cyclohexenyl, phenyl, pyridyl wherein the nitrogen is at the 1-, 2-, or 3-position, and a 6-membered heterocyclic ring having one heteroatom selected from the group consisting of sulfur and oxygen at the 1-, 2-, or 3-position and nitrogen at the 1-, 2-, 3-, or 4-position, or wherein one carbon on the heterocyclic ring is optionally substituted with ═O; and wherein one of A or Z is a heterocyclic ring;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

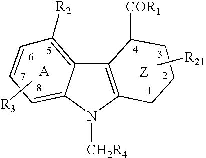

wherein:
Z is selected from the group consisting of cyclohexenyl and phenyl;
R₂₁ is a non-interfering substituent;
R₁ is —NHNH₂ or —NH₂;
R₂ is selected from the group consisting of —OH and —O(CH₂)ₘR₅; wherein R₅ is selected from the group consisting of H, —CO₂H, —CONH₂, —CO₂(C₁ -C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

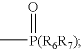

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; R₉ and R₁₀ are each independently selected from the group consisting of H, —CF₃, phenyl, —(C₁-C₄)alkyl, —(C₁-C₄)alkylphenyl, and -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, phenyl substituted with —(C₁-C₆)alkyl, halo, —CF₃, —OCF₃, —(C₁-C₄)alkoxy, —CN, —(C₁-C₄)alkylthio, -phenyl(C₁-C₄)alkyl, —(C₁-C₄)alkylphenyl, phenyl, phenoxy and naphthyl;
and pharmaceutically acceptable salts, solvates, prodrug derivatives, racemates, tautomers, or optical isomers thereof.

In certain embodiments, sPLA₂ inhibitors for use in the current invention are selected from the group consisting of: {9-[(phenyl)methyl]-5-carbamoylcarbazol-4-yl}oxyacetic acid; 9-benzyl-5,7-dimethoxy-1,2,3,4-tetrahydrocarbazole-4-carboxylic acid hydrazide; 9-benzyl-5,7-dimethoxy-1,2,3,4-tetrahydrocarbazole-4-carboxamide; [9-benzyl-4-carbamoyl-7-methoxy-1,2,3,4-tetrahydrocarbazol-5-yl]oxyacetic acid; [9-benzyl -4-carbamoyl-7-methoxycarbazol-5-yl]oxyacetic acid; methyl [9-benzyl-4-carbamoyl-7-methoxycarbazol-5-yl]oxyacetic acid; 9-benzyl-7-methoxy-5-cyanomethyloxy-1,2,3,4-tetrahydrocarbazole-4-carboxamide; 9-benzyl-7-methoxy-5-(1H-tetrazol-5-yl-methyl)oxy)-1,2,3,4-tetrahydrocarbazole-4-carboxamide; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazol-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-4-carbamoyl-8-chloro-1,2,3,4-tetrahydrocarbazol-5-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; {9-[(3-fluorophenyl)methyl]-5-carbamoylcarbazol-4-yl}oxyacetic acid; {9-[(3-chlorophenyl)methyl]-5-carbamoylcarbazol-4-yl}oxyacetic acid; {9-[(3-phenoxyphenyl)methyl]-5-carbamoylcarbazol-4-yl}oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(3-trifluoromethylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(2-methylphenyl)methyl]-5-carbamoylcarbazol-4-yl}oxyacetic acid; {9-[(3-methylphenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(3-trifluoromethoxyphenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazole-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-5-carbamoyl-1-fluorocarbazol-4-yl]oxyacetic acid; [9-benzyl-4-carbamoyl-8-chloro-1,2,3,4-tetrahydrocarbazol-5-yl]oxyacetic acid; [9-benzyl-5-carbamoyl-1-chlorocarbazol-4-yl]oxyacetic acid; [9-[(Cyclohexyl)methyl]-5-carbamoylcarbazol-4-yl]oxyacetic acid; [9-[(Cyclopentyl)methyl]-5-carbamoylcarbazol-4-yl]oxyacetic acid; [5-carbamoyl-9-(phenylmethyl)-2-(2-thienyl)carbazol-4-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; (R,S)-(9-benzyl-4-carbamoyl-1-oxo-3-thia-1,2,3,4-tetrahydrocarbazol-5-yl)oxyacetic acid; (R,S)-(9-benzyl-4-carbamoyl-3-thia-1,2,3,4-tetrahydrocarbazol-5-yl)oxyacetic acid; 2-(4-oxo-5-carboxamido-9-benzyl-9H-pyrido[3,4-b]indolyl)acetic acid chloride; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-betacarbolin-5-yl)oxy]acetic acid; 2-[4-oxo-5-carboxamido-9-(2-methylbenzyl)-9H-pyrido[3,4-b]indolyl]acetic acid; 2-[4-oxo-5-carboxamido-9-(3-methylbenzyl)-9H-pyrido[3,4-b]indolyl]acetic acid; 2-[4-oxo-5-carboxamido-9-(4-methylbenzyl)-9H-pyrido[3,4-b]indolyl]acetic acid; 2-[4-oxo-5-carboxamido-9-(4-tert-butylbenzyl)-9H-pyrido[3,4-b]indolyl]acetic acid; 2-[4-oxo-5-carboxamido-9-pentafluorobenzyl-9H-pyrido[3,4-b]indolyl]acetic acid; 2-[4-oxo-5-carboxamido-9-(2-fluorobenzyl)-9H-pyrido[3,4-b]indolyl]acetic acid; 2-[4-oxo-5-carboxamido-9-(3-fluorobenzyl)-9H-pyrido[3,4-b]indolyl]acetic acid; 2-[4-oxo-5-carboxamido-9-(4-fluorobenzyl)-9H-pyrido[3,4-b]indolyl]acetic acid; 2-[4-oxo-5-carboxamido-9-(2,6-difluorobenzyl)-9H-pyrido[3,4-b]indolyl]acetic acid; 2-[4-oxo-5-carboxamido-9-(3,4-difluorobenzyl)-9H-pyrido[3,4-b]indolyl]acetic acid; 2-[4-oxo-5-carboxamido-9-(2,5-difluorobenzyl)-9H-pyrido[3,4-b]indolyl]acetic acid; 2-[4-oxo-5-carboxamido-9-(3,5-difluorobenzyl)-9H-pyrido[3,4-b]indolyl]acetic acid; 2-[4-oxo-5-carboxamido-9-(2,4-difluorobenzyl)-9H-pyrido[3,4-b]indolyl]acetic acid; 2-[4-oxo-5-carboxamido-9-(2,3-difluorobenzyl)-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[3-(trifluoromethyl)benzyl]-9H-pyrido[3,4-b]indolyl]acetic acid; 2-[4-oxo-5-carboxamido-9-[4-(trifluoromethyl)benzyl]-9H-pyrido[3,4-b]indolyl]acetic acid; 2-[4-oxo-5-carboxamido-9-[3,5-bis(trifluoromethyl)benzyl]-9H-pyrido[3,4-b]indolyl]acetic acid; 2-[4-oxo-5-carboxamido-9-[2,4-bis(trifluoromethyl)benzyl]-9H-pyrido[3,4-b]indolyl]acetic acid; 2-[4-oxo-5-carboxamido-9-(a-methylnaphthyl)-9H-pyrido[3,4-b]indolyl]acetic acid; 2-[4-oxo-5-carboxamido-9-(b-methylnaphthyl)-9H-pyrido[3,4-b]indolyl]acetic acid; 2-[4-oxo-5-carboxamido-9-(3,5-dimethylbenzyl)-9H-pyrido[3,4-b]indolyl]acetic acid; 2-[4-oxo-5-carboxamido-9-(2,4-dimethylbenzyl)-9H-pyrido[3,4-b]indolyl]acetic acid; 2-[4-oxo-5-carboxamido-9-(2-phenylbenzyl)-9H-pyrido[3,4-b]indolyl]acetic acid; 2-[4-oxo-5-carboxamido-9-(3-phenylbenzyl)-9H-pyrido[3,4-b]indolyl]acetic acid; 2-[4-oxo-5-carboxamido-9-(4-phenylbenzyl)-9H-pyrido[3,4-b]indolyl]acetic acid; 2-[4-oxo-5-carboxamido-9-(1-fluorenylmethy)-9H-pyrido[3,4-b]indolyl]acetic acid; 2-[4-oxo-5-carboxamido-9-(2-fluoro-3-methylbenzyl)-9H-pyrido[3,4-b]indolyl]acetic acid; 2-[4-oxo-5-carboxamido-9-(3-benzoylbenzyl)-9H-pyrido[3,4-b]indolyl]acetic acid; 2-[4-oxo-5-carboxamido-9-(2-phenoxybenzyl)-9H-pyrido[3,4-b]indolyl]acetic acid; 2-[4-oxo-5-carboxamido-9-(3-phenoxybenzyl)-9H-pyrido[3,4-b]indolyl]acetic acid; 2-[4-oxo-5-carboxamido-9-(4-phenoxybenzyl)-9H-pyrido[3,4-b]indolyl]acetic acid; 2-[4-oxo-5-carboxamido-9-[3-[2-(fluorophenoxy)benzyl]]-9H-pyrido[3,4-b]indolyl]acetic acid; 2-[4-oxo-5-carboxamido-9-[3-[4-(fluorophenoxy)benzyl]]-9H-pyrido[3,4-b]indolyl]acetic acid; 2-[4-oxo-5-carboxamido-9-[2-fluoro-3-(trifluoromethyl)benzyl]-9H-pyrido[3,4-b]indolyl]acetic acid; 2-[4-oxo-5-carboxamido-9-[2-fluoro-4-(trifluoromethyl)benzyl]-9H-pyrido[3,4-b]indolyl]acetic acid; 2-[4-oxo-5-carboxamido-9-[2-fluoro-5-(trifluoromethyl)benzyl]-9H-pyrido[3,4-b]indolyl]acetic acid; 2-[4-oxo-5-carboxamido-9-[3-fluoro-5-(trifluoromethyl)benzyl]-9H-pyrido[3,4-b]indolyl]acetic acid; 2-[4-oxo-5-carboxamido-9-[4-fluoro-2-(trifluoromethyl)benzyl]-9H-pyrido[3,4-b]indolyl]acetic acid; 2-[4-oxo-5-carboxamido-9-[4-fluoro-3-(trifluoromethyl)benzyl]-9H-pyrido[3,4-b]indolyl]acetic acid; 2-[4-oxo-5-carboxamido-9-[2-fluoro-6-(trifluoromethyl)benzyl]-9H-pyrido[3,4-b]indolyl]acetic acid; 2-[4-oxo-5-carboxamido-9-(2,3,6-trifluorobenzyl)-9H-pyrido[3,4-b]indolyl]acetic acid; 2-[4-oxo-5-carboxamido-9-(2,3,5-trifluorobenzyl )-9H-pyrido[3,4-b]indolyl]acetic acid; 2-[4-oxo-5-carboxamido-9-(2,4,5-trifluorobenzyl)-9H-pyrido[3,4-b]indolyl]acetic acid; 2-[4-oxo-5-carboxamido-9-(2,4,6-trifluorobenzyl)-9H-pyrido[3,4-b]indolyl]acetic acid; 2-[4-oxo-5-carboxamido-9-(2,3,4-trifluorobenzyl)-9H-pyrido[3,4-b]indolyl]acetic acid; 2-[4-oxo-5-carboxamido-9-(3,4,5-trifluorobenzyl)-9H-pyrido[3,4-b]indolyl]acetic acid; 2-[4-oxo-5-carboxamido-9-[3-(trifluoromethoxyl)benzyl]-9H-pyrido[3,4-b]indolyl]acetic acid; 2-[4-oxo-5-carboxamido-9-[4-(trifluoromethoxyl)benzyl]-9H-pyrido[3,4-b]indolyl]acetic acid; 2-[4-oxo-5-carboxamido-9-[4-methoxy(tetrafluoro)benzyl]-9H-pyrido[3,4-b]indolyl]acetic acid; 2-[4-oxo-5-carboxamido-9-(2-methoxybenzyl)-9H-pyrido[3,4-b]indolyl]acetic acid; 2-[4-oxo-5-carboxamido-9-(3-methoxybenzyl)-9H-pyrido[3,4-b]indolyl]acetic acid; 2-[4-oxo-5-carboxamido-9-(4-methoxybenzyl)-9H-pyrido[3,4-b]indolyl]acetic acid; 2-[4-oxo-5-carboxamido-9-(4-ethylbenzyl)-9H-pyrido[3,4-b]indolyl]acetic acid; 2-[4-oxo-5-carboxamido-9-(4-isopropylbenzyl)-9H-pyrido[3,4-b]indolyl]acetic acid; 2-[4-oxo-5-carboxamido-9-(3,4,5-trimethoxybenzyl)-9H-pyrido[3,4-b]indolyl]acetic acid; 2-[4-oxo-5-carboxamido-9-(3,4-methylenedioxybenzyl)-9H-pyrido[3,4-b]indolyl]acetic acid; 2-[4-oxo-5-carboxamido-9-(4-methoxy-3-methylbenzyl)-9H-pyrido[3,4-b]indolyl]acetic acid; 2-[4-oxo-5-carboxamido-9-(3,5-dimethoxybenzyl)-9H-pyrido[3,4-b]indolyl]acetic acid; 2-[4-oxo-5-carboxamido-9-(2,5-dimethoxybenzyl)-9H-pyrido[3,4-b]indolyl]acetic acid; 2-[4-oxo-5-carboxamido-9-(4-ethoxybenzyl)-9H-pyrido[3,4-b]indolyl]acetic acid; 2-[4-oxo-5-carboxamido-9-(cyclohexylmethyl)-9H-pyrido[3,4-b]indolyl]acetic acid; 2-[4-oxo-5-carboxamido-9-(cyclopentylmethyl)-9H-pyrido[3,4-b]indolyl]acetic acid; 2-[4-oxo-5-carboxamido-9-ethyl-9H-pyrido[3,4-b]indolyl]acetic acid; 2-[4-oxo-5-carboxamido-9-(1-propyl)-9H-pyrido[3,4-b]indolyl]acetic acid; 2-[4-oxo-5-carboxamido-9-(2-propyl)-9H-pyrido[3,4-b]indolyl]acetic acid; 2-[4-oxo-5-carboxamido-9-(1-butyl)-9H-pyrido[3,4-b]indolyl]acetic acid; 2-[4-oxo-5-carboxamido-9-(2-butyl)-9H-pyrido[3,4-b]indolyl]acetic acid; 2-[4-oxo-5-carboxamido-9-isobutyl-9H-pyrido[3,4-b]indolyl]acetic acid; 2-[4-oxo-5-carboxamido-9-[2-(1-phenylethyl)]-9H-pyrido[3,4-b]indolyl]acetic acid; 2-[4-oxo-5-carboxamido-9-[3-(1-phenylpropyl)]-9H-pyrido[3,4-b]indolyl]acetic acid; 2-[4-oxo-5-carboxamido-9-[4-(1-phenylbutyl)]-9H-pyrido[3,4-b]indolyl]acetic acid; 2-[4-oxo-5-carboxamido-9-(1-pentyl)-9H-pyrido[3,4-b]indolyl]acetic acid; 2-[4-oxo-5-carboxamido-9-(1-hexyl)-9H-pyrido[3,4-b]indolyl]acetic acid; 4-[(9-benzyl-4-carbamoyl-1,2,3,4-tetrahydrocarbazol-6-yl)oxy]butyric acid; 3-[(9-benzyl-4-carbamoyl-1,2,3,4-tetrahydrocarbazol-6-yl)oxy]propylphosphonic acid; 2-[(9-benzyl-4-carbamoyl-1,2,3,4-tetrahydrocarbazol-6-yl)oxy]methylbenzoic acid; 3-[(9-benzyl-4-carbamoyl-7-n-octyl-1,2,3,4-tetrahydrocarbazol-6-yl)oxy]propylphosphonic acid; 4-[(9-benzyl-4-carbamoyl-7-ethyl-1,2,3,4-tetrahydrocarbazol-6-yl)oxy]butyric acid; 3-[(9-benzyl-4-carbamoyl-7-ethyl-1,2,3,4-tetrahydrocarbazol-6-yl)oxy]propylphosphonic acid; 3-[(9-benzyl-4-carbamoyl-7-ethyl-1,2,3,4-tetrahydrocarbazol-6-yl)oxy]propylphosphonic acid; (S)-(+)-4-[(9-benzyl-4-carbamoyl-7-ethyl-1,2,3,4-tetrahydrocarbazol-6-yl)oxy]butyric acid; 4-[9-benzyl-4-carbamoyl-6-(2-cyanoethyl)-1,2,3,4-tetrahydrocarbazol-6-yl]oxybutyric acid; 4-[9-benzyl-4-carboxamido-7-(2-phenylethyl)-1,2,3,4-tetrahydrocarbazol-6-yl]oxybutyric acid; 4-[9-benzyl-4-carboxamidocarbazol-6-yl]oxybutyric acid; methyl 2-[(9-benzyl-4-carbamoyl-1,2,3,4-tetrahydrocarbazol-6-yl)oxy]methylbenzoate; 4-[9-benzyl-4-carbamoyl-7-(2-cyanoethyl)-1,2,3,4-tetrahydrocarbazol-6-yl]oxybutyric acid; 9-benzyl-7-methoxy-5-cyanomethyloxy-1,2,3,4-tetrahydrocarbazole-4-carboxamide; [9-benzyl-4-carbamoyl-8-methyl-carbazole-5-yl]oxyacetic acid; and [9-benzyl-4-carbamoyl-carbazole-5-yl]oxyacetic acid, or pharmaceutically acceptable salts, solvates, prodrug derivatives, racemates, tautomers, or optical isomers thereof.

In certain preferred embodiments, an sPLA₂ inhibitor compound for use in the present invention is ((3-(2-Amino-1,2-dioxoethyl)-2-ethyl-1-(phenylmethyl)-1H-indol-4-yl)oxy)acetic acid, also referred to herein as compound A-001. Compound A-001, which is also referred to in the art as S-5920 or LY315920, has the structure:

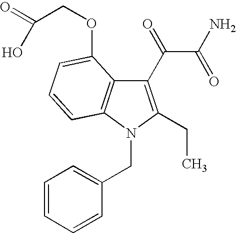

A-001 competitively inhibits sPLA₂.

In certain other preferred embodiments, an sPLA₂ inhibitor compound for use in the present invention is [[3-(2-Amino-1,2-dioxoethyl)-2-ethyl-1-(phenylmethyl)-1H-indol-4-yl]oxy]acetic acid methyl ester, also referred to herein as compound A-002. Compound A-002 has the structure:

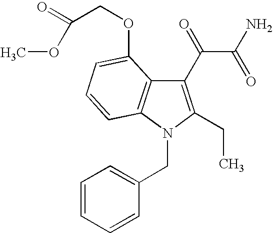

Compound A-002, which is sometimes referred to in the art as S-3013 or LY333013, is an A-001 prodrug that is hydrolyzed to compound A-001 following administration to a subject.

In certain other preferred embodiments, an sPLA₂ inhibitor for use in the present invention is {9-[(phenyl)methyl]-5-carbamoylcarbazol-4-yl}oxyacetic acid, also referred to herein as compound A-003 or LY433771. Compound A-003 has the structure:

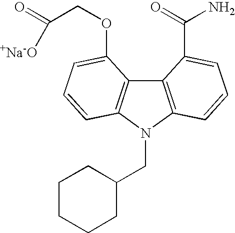

In still other preferred embodiments, an sPLA₂ inhibitor compound for use in the present invention is ((3-(2-amino-1,2-dioxoethyl)-2-methyl-1-(phenylmethyl)-1H-indol-4-yl)oxy)acetic acid N-morpholino ethyl ester, also referred to herein as compound 421079. Compound 421079 has the structure:

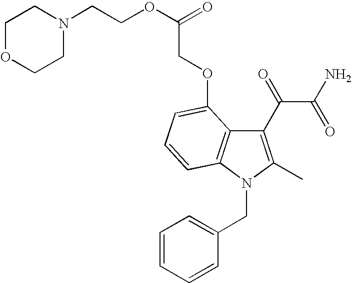

Like A-002, compound 421079 is a prodrug of A-001.

In yet other preferred embodiments, the compound is the sodium salt of compound A-001, compound A-002, compound A-003, or compound 421079.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Depletion of sPLA₂ from Human Serum

To generate antibody for depletion of sPLA₂ from human serum, sPLA₂ coating antibody (1.84 mg/ml, Cayman, Catalog No. 10009887) was diluted to 1 μg/ml in 3 ml 0.6 M sodium citrate, pH >7.5. 2 ml of 1 μg/ml antibody was added to 0.04 g of UltraLink Biosupport beads (Pierce, 53110) in a 15 ml centrifuge tube, vortexed, rotated for 1 hour at room temperature, and centrifuged at 1,200×g at room temperature for 5-10 minutes. Supernatant was removed, and the pellet was resuspended in 4 ml 1 M Tris, pH 8.0 quench solution. The suspension was vortexed, rotated for 2.5 hours at room temperature, and centrifuged at 1,200×g at room temperature for 5-10 minutes. Supernatant was removed, and the pellet was resuspended in 4 ml PBS wash solution, vortexed, and rotated for 15 minutes at room temperature. The suspension was then centrifuged at 1,200×g at room temperature for 5-10 minutes, and the supernatant was removed. The pellet was resuspended in 1 M NaCl PBS, vortexed, rotated for 15 minutes at room temperature, and centrifuged at 1,200×g at room temperature for 5-10 minutes. Supernatant was removed, and the pellet was resuspended in 4 ml PBS, vortexed, rotated for 15 minutes, and centrifuged at 1,200×g at room temperature for 5-10 minutes. The PBS wash step was repeated two more times, and the pellet was resuspended in PBS for storage at 2-8° C.

For depletion of sPLA₂, the stored antibody solution prepared in the preceding step was centrifuged at 1,200×g at room temperature for 5-10 minutes, the supernatant was removed, and the pellet was resuspended in 10 to 15 ml of human serum (Bioreclamation, HMSRM Lot #BRH137802). The suspension was gently vortexed, rotated for 1 hour at room temperature, and centrifuged at 1,200×g at room temperature for 5-10 minutes. The supernatant was collected and stored at 2-8° C.

### Example 2

### Initial Testing of sPLA₂-Depleted Serum

20 ng sPLA₂ was reconstituted in 50 μl EIA buffer plus 5% mouse serum to generate a 400 ng/ml stock. EIA buffer was prepared by diluting the contents of one vial of EIA Buffer concentrate from the Cayman kit with 90 ml of deionized water free of trace organic contaminants (UltraPure). The vial was rinsed to remove any salts that may have precipitated. The 400 ng/ml sPLA2 stock was used to generate three sets of standards utilizing the following three diluents: buffer only, neat human serum (“normal serum”), and neat human serum from which sPLA₂ had been depleted using sPLA₂ antibody (see Example 1) (“purified serum”).

Each set of standards (buffer, purified serum, normal serum) contained eight samples: 4.00 ng/ml, 2.00 ng/ml, 1.00 ng/ml, 0.50 ng/ml, 0.25 ng/ml, 0.13 ng/ml, 0.06 ng/ml, and 0 ng/ml. The 0 ng/ml sample (“standard H”) contained diluent only. The dilution strategy for the other standard samples was as follows:
5 μl of 400 ng/ml stock into 495 μl diluent to generate 4.00 ng/ml standard A;
100 l 4.00 ng/ml standard A into 100 μl diluent to generate 2.00 ng/ml standard B; 100 μl 2.00 ng/ml standard B into 100 μl diluent to generate 1.00 ng/ml standard C;
100 μl 1.00 ng/ml standard C into 100 μl diluent to generate 0.50 ng/ml standard D;
100 μl 0.50 ng/ml standard D into 100 μl diluent to generate 0.25 ng/ml standard E;
100 μl 0.25 ng/ml standard E into 100 μl diluent to generate 0.13 ng/ml standard F; and
100 μl 0.13 ng/ml standard F into 100 μl diluent to generate 0.06 ng/ml standard G.
   Each sample was loaded in duplicate onto the 96-well EIA plate from the Cayman kit at a volume of 30 μl/well.

Rather than the 10 ml of EIA buffer normally used with the Cayman kit, conjugate (100 dtn) was reconstituted in 20 ml of EIA buffer for a final conjugate concentration of 5 dtn/ml. Reconstituted conjugate was added to each well of the EIA plate at a volume of 60 μl/well, and the plate was covered with plastic film and incubated overnight at 2-8° C.

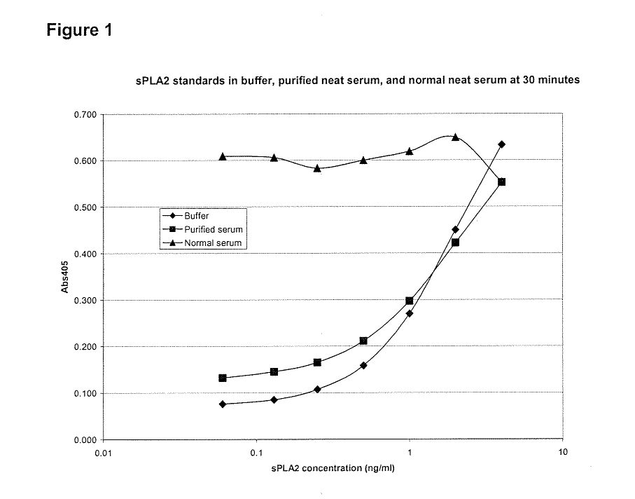

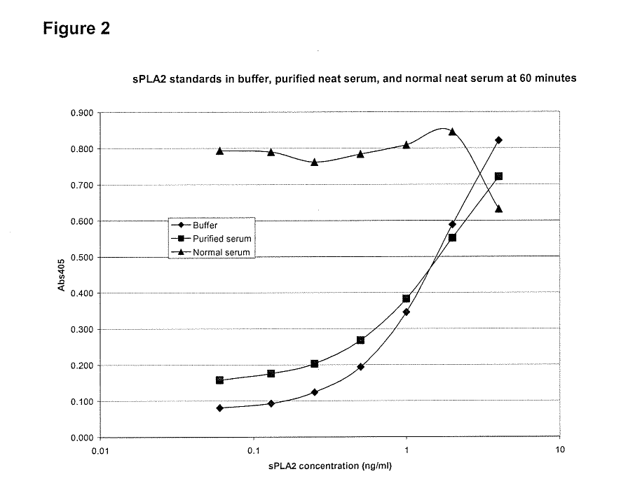

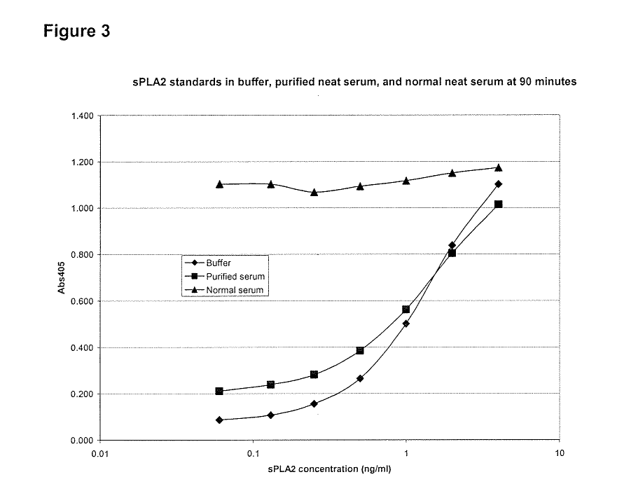

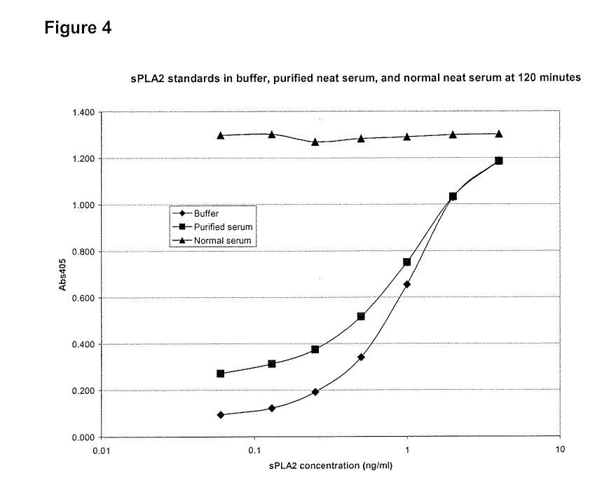

After incubation, the EIA plate was washed five to six times with buffer. Reconstituted Ellman's reagent (5 dtn/ml) was added to each well at a volume of 30 μl/well, and the plate was developed at room temperature on a shaker. Absorbance at 405 nm was measured at 30, 60, 90, and 120 minutes in order to determine the optimal development time, and standard curves were generated. As shown in Tables 1-4 and FIGS. 1-4, purified serum standards performed similarly to standards diluted in buffer only, while normal serum demonstrated a high level of sPLA₂. In addition, the results showed that 1 to 1.5 hours was the optimal development time, with 2 hours also providing acceptable results.

### Example 3

### Determination of Detection Limits for Depleted Serum Assay

400 ng/ml sPLA₂ stock was used to generate four sets of standards utilizing the following four diluents: normal serum, 10% purified human serum/5% mouse serum, 1% purified human serum/5% mouse serum, and 0.1% purified human serum/5% mouse serum.

Each set of standards (normal, 10% purified, 1% purified, and 0.1% purified) contained eight samples: 4.00 ng/ml, 2.00 ng/ml, 1.00 ng/ml, 0.50 ng/ml, 0.25 ng/ml, 0.13 ng/ml, 0.06 ng/ml, and 0 ng/ml, with the 0 ng/ml sample containing diluent only. The dilution strategy for the other standard samples was identical to that set forth above, but with a final sample volume of 80 μl instead of 100 μl. Each sample was loaded in duplicate onto the 96-well EIA plate from the Cayman kit at a volume of 30 μl/well.

Reconstituted conjugate (5 dtn/ml) was added to each well of the EIA plate at a volume of 60 μl/well, and the plate was covered with plastic film and incubated overnight at 2-8° C.

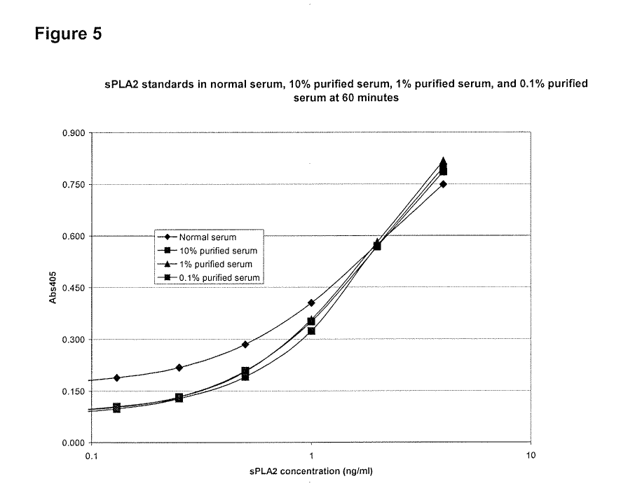

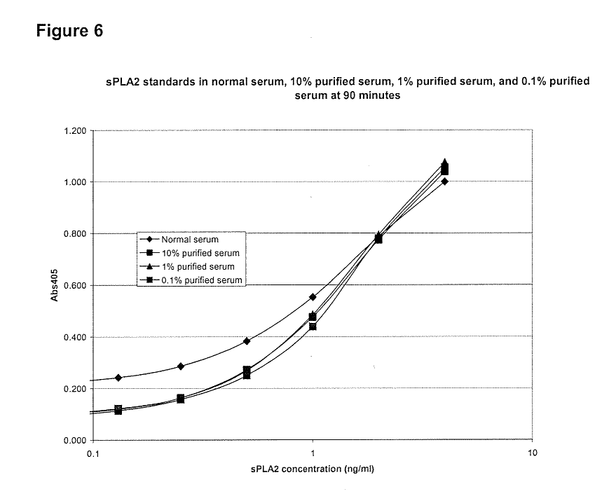

After incubation, the EIA plate was washed five to six times with buffer. Reconstituted Ellman's reagent (5 dtn/ml) was added to each well at a volume of 30 μl/well, and the plate was developed at room temperature on a shaker. Absorbance at 405 nm was measured at 60, 90, 105, and 120 minutes, and standard curves were generated. As shown in Tables 5-6 and FIGS. 5-6, the range of detection for the assay was at least 0.05 ng/ml to 4000 ng/ml.

### Example 4

### Prevalidation of Depleted Serum Assay: Calculation of Recovery Values

400 ng/ml sPLA₂ stock was used to generate four sets of standard curve samples utilizing the following diluents: purified human serum, 10% purified human serum/5% mouse serum, 1% purified human serum/5% mouse serum, and 0.1% purified human serum/5% mouse serum. Each set of standards contained eight samples each: 6.00 ng/ml, 4.00 ng/ml, 2.00 ng/ml, 1.00 ng/ml, 0.50 ng/ml, 0.17 ng/ml, 0.06 ng/ml, and 0 ng/ml, with the 0 ng/ml sample containing diluent only.

A 200 ng/ml sPLA₂ stock was generated by diluting sPLA₂ into 100 μl EIA and 5% mouse serum, and this stock was diluted to 20 ng/ml by adding 900 μl purified human serum, 10% purified human serum/5% mouse serum, 1% purified human serum/5% mouse serum, or 0.1% purified human serum/5% mouse serum. A set of quality control samples were then generated by diluting these 20 ng/ml samples as follows:
1.4 μl of 20 ng/ml solution into 12.6 μl diluent to generate 2.00 ng/ml (“high”) quality control;
0.7 μl of 20 ng/ml solution into 13.3 μl diluent to generate 1.00 ng/ml (“medium”) quality control; and
0.35 μl of 20 ng/ml solution into 13.65 μl diluent to generate 0.50 ng/ml (“low”) quality control.

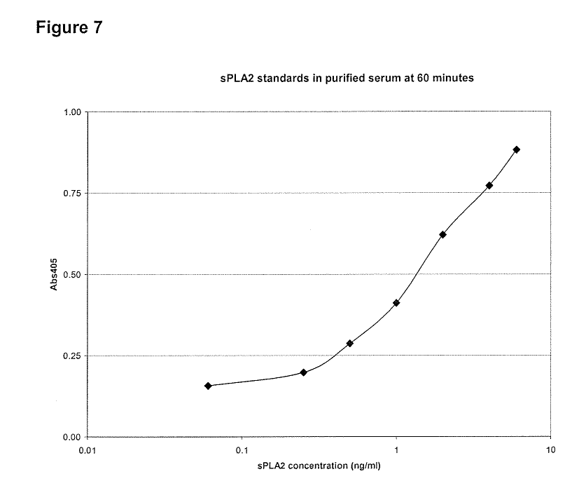

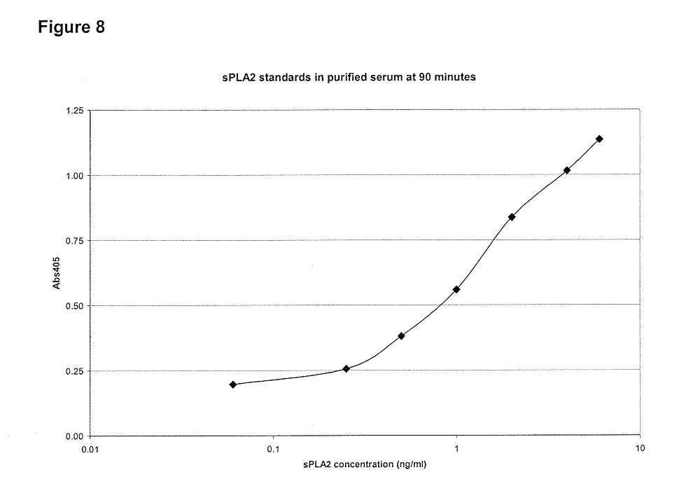

All samples, including quality controls, were loaded in duplicate onto the 96-well EIA plate from the Cayman kit at a volume of 30 μl/well. Reconstituted conjugate (5 dtn/ml) was added to each well of the EIA plate at a volume of 60 μl/well, and the plate was covered with plastic film and incubated overnight at 2-8° C. After incubation, the EIA plate was washed five to six times with buffer. Reconstituted Ellman's reagent (5 dtn/ml) was added to each well at a volume of 30 μl/well, and the plate was developed at room temperature on a shaker. Absorbance at 405 nm was measured at 60 and 90 minutes. Data for standard curve samples at 60 minutes and 90 minutes (Tables 7 and 8, respectively) was used to generate standard curves for purified serum (FIGS. 7 and 8). Percent recovery values were determined for the quality control samples using these standard curves (Tables 9 and 10). Percent recovery values ranged from 91 to 93% at 1 hour and from 91% to 96% at 1.5 hours.

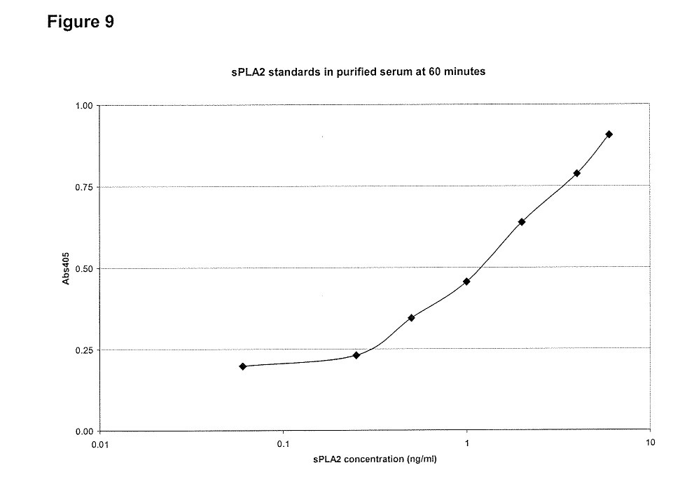

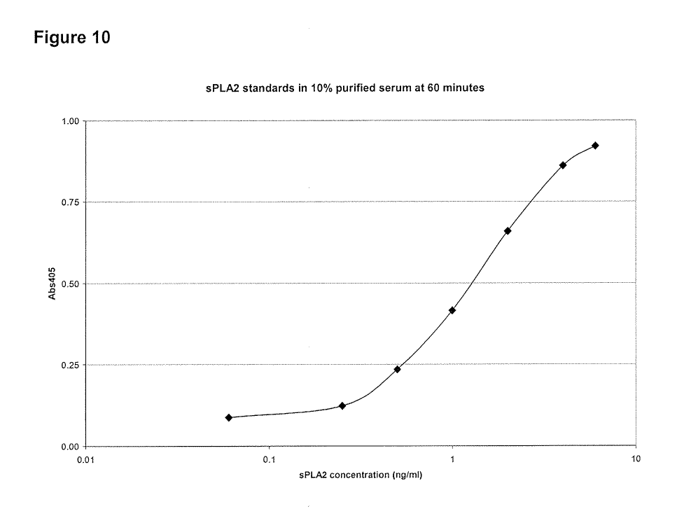

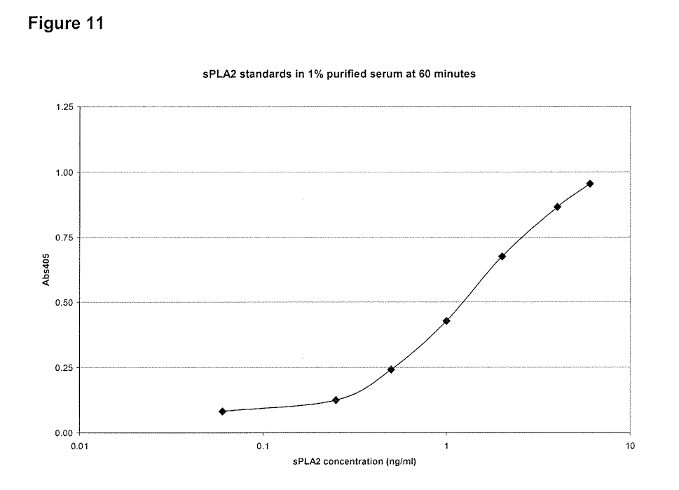

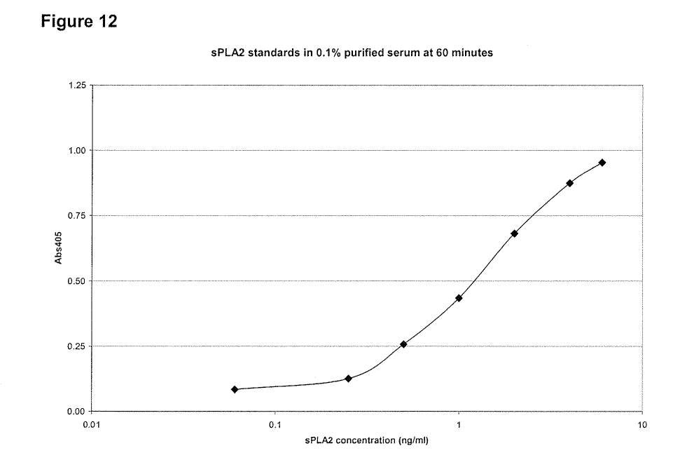

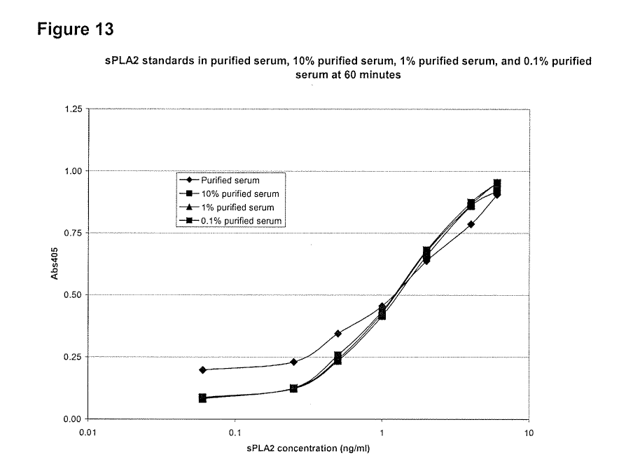

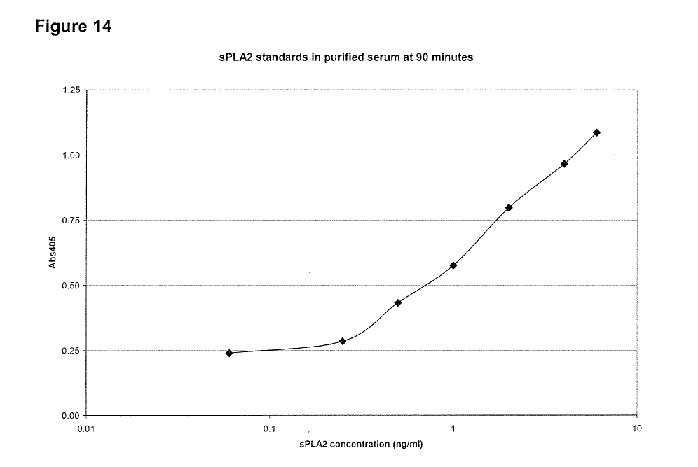

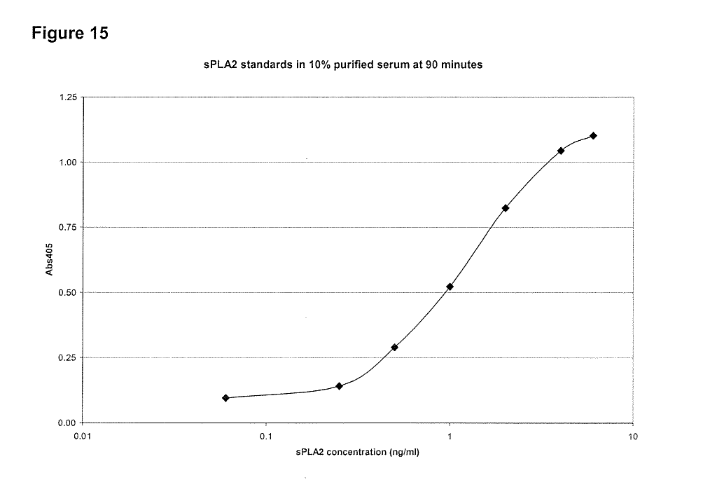

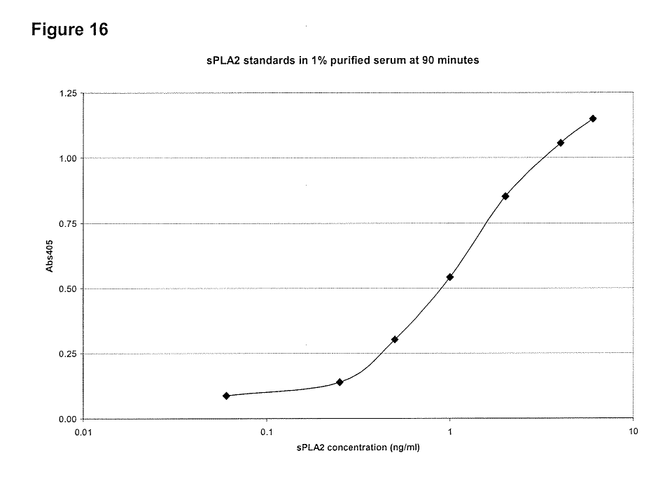

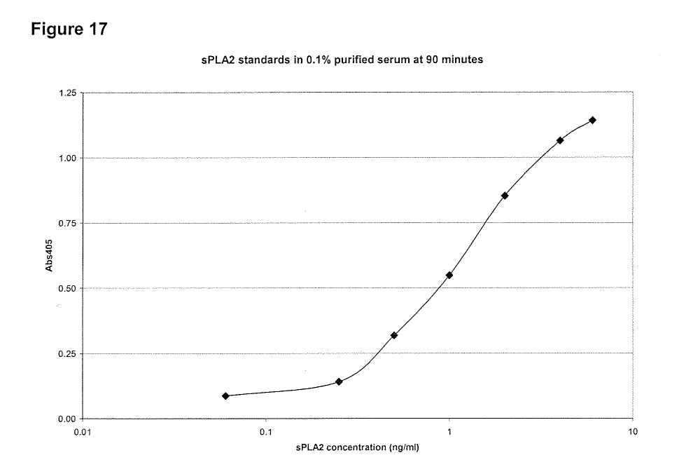

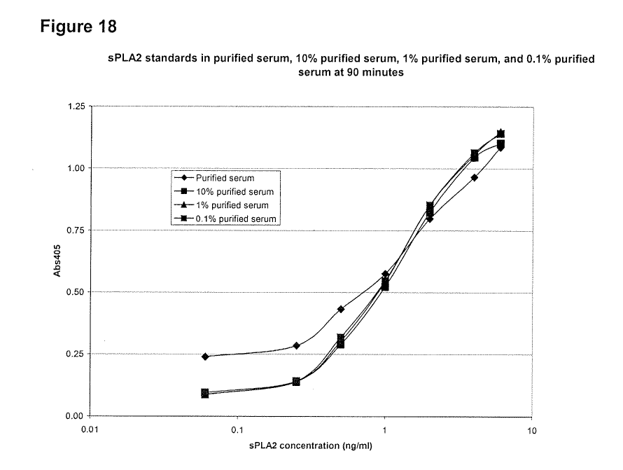

The percent recovery experiment was repeated using a fresh batch of purified serum. Eight standard curve samples (6, 4, 2, 1, 0.5, 0.25, 0.06, and 0 ng/ml) and three quality control samples (2, 1, and 0.5 ng/ml) were generated using each diluent (purified serum, 10% purified serum, 1% purified serum, and 0.1% purified serum), and all samples were run in duplicate with a 60 minute or 90 minute development time. Data for standard curve samples at 60 minutes and 90 minutes (Tables 11-14 and 16-19, respectively) was used to generate standard curves for purified serum, 10% purified serum, 1% purified serum, and 0.1% purified serum, as well as a combined standard curve incorporating data for all four diluents (FIGS. 9-13, respectively, for 60 minute samples; FIGS. 14-18, respectively, for 90 minute samples). Percent recovery values were determined for the quality control samples using these standard curves (Tables 11-14 for 60 minute samples, Tables 16-19 for 90 minute samples). Recovery results are summarized in Table 15 (60 minutes), Table 20 (90 minutes), and Table 21 (60 and 90 minutes).

### Example 5

### Protocol for Depleted Serum Assay Sample Preparation

The following sample preparation protocol was developed based on the experiments set forth in Examples 2-4, above.

Diluents for use in preparing standard curve, quality control, and test samples (when dilution of test samples is necessary) contain purified human serum from which sPLA2 has been depleted. Purified human serum may comprise from about 0.1% to 100% of diluent volume. In certain embodiments, the diluents may also contain mouse serum from about 0.1 to about 10%. The percentage of purified human serum in the diluent may be varied depending on the expected concentration of sPLA2 in the test sample, with a higher percentage of purified human serum being utilized when measuring test samples that are expected to have lower concentrations of sPLA2. In certain embodiments, standard curve samples may be prepared using the following diluents:

| Range of expected level of sPLA₂ | |
|---|---|
| in the serum (ng/ml) | Standard diluent |
| 0.25-4 | Human purified serum, 5% mouse |
| | serum (Cayman Catalog No. 160502 |
| | or equivalent) |
| 2.5-40 | 10% purified human serum, 5% |
| | mouse serum, EIA buffer (Cayman |
| | Catalog No. 400060 or equivalent) |
| 25-400 | 1% purified human serum, 5% mouse |
| | serum, EIA buffer |
| 250-4000 | 0.1% purified human serum, 5% |
| | mouse serum, EIA buffer |

sPLA₂ (Cayman Catalog No. 485004 or equivalent) is reconstituted in EIA buffer plus 0-10% mouse serum to generate an sPLA2 stock solution. This stock solution is then used to generate standard curve samples with sPLA2 concentrations ranging from 0 to around 8 ng/ml in an appropriate diluent. For example, in one embodiment, a set of standard curve samples may have sPLA2 concentrations of 6, 4, 2, 1, 0.5, 0.17, 0.06, and 0 ng/ml. In such an embodiment, standard curve samples may be generated as follows:

| | Final sPLA₂ | | |
|---|---|---|---|
| | concentration | | |
| Tube | (ng/ml) | μl diluent | sPLA₂ |
| 1 | 6.00 | 345 | 5.25 μl of 400 ng/ml |
| | | | stock |
| 2 | 4.00 | 50 | 100 μl from tube 1 |
| 3 | 2.00 | 75 | 75 μl from tube 2 |
| 4 | 1.00 | 75 | 75 μl from tube 3 |
| 5 | 0.50 | 75 | 75 μl from tube 4 |
| 6 | 0.17 | 100 | 50 μl from tube 5 |
| 7 | 0.06 | 96 | 54 μl from tube 6 |
| 8 | 0 | 75 | 0 μl |

Test samples from subjects are diluted as follows:

| Diluent | Dilution of test sample |
|---|---|
| Purified serum | No dilution required |
| 10% purified serum | Dilute 1:10 |
| 1% purified serum | Dilute 1:100 |
| 0.1% purified serum | Dilute 1:1000 |

In addition to standard curve samples, a set of quality control samples is generated for each diluent. Quality control samples contain a known concentration of sPLA₂, and are used to verify the accuracy of the standard curve. For each assay, multiple quality control samples covering a range of concentrations should be utilized. In one embodiment, three quality control samples may be used, with a first sample having a relatively high sPLA₂ concentration, a second sample having a relatively low sPLA₂ concentration, and a third sample with an sPLA₂ concentration between those of the first two samples. For example, in these embodiments, the sPLA₂ concentration of the high, medium, and low quality control samples may be 2, 1, and 0.5 ng/ml, respectively.

### Example 6

### Effect of sPLA₂ Inhibitor Administration on sPLA₂ Levels

84 subjects with stable coronary artery disease were randomized to receive placebo or A-002 via oral administration twice a day over a four week administration period. This ITT population included 24 subjects with diabetes, 32 subjects with metabolic syndrome, and 75 subjects that were receiving statin treatment before and during the trial. Subjects with metabolic syndrome were identified as those subjects meeting three of the five following criteria: 1) abdominal obesity (waist circumference >35 inches in women, >40 inches in men); 2) low HDL levels (<50 mg/dL in women, <40 mg/dL in men); 3) high blood pressure (≧130/85 mm Hg) or current treatment with antihypertensive medication; 4) high triglyceride levels (≧150 mg/dL); and 5) impaired fasting glucose (blood glucose levels of 110-126 mg/dL).

A-002 was administered at 50 mg, 100 mg, 250 mg, or 500 mg dosages. Serum sPLA₂ levels were measured at the outset of the trial and at the end of weeks two and four using the novel sPLA₂ assay disclosed herein. Administration of A-002 decreased serum sPLA₂ levels in the ITT population at all dosages tested in a dose-independent manner (Table 21). This decrease was observed in diabetic and non-diabetic subpopulations (Tables 22 and 23), as well as in the metabolic syndrome subpopulation (Table 26). Likewise, the decrease was observed in both statin and non-statin subpopulations (Tables 24 and 25). These results establish that A-002 is effective at decreasing sPLA₂ levels in a wide range of subjects exhibiting conditions associated with sPLA₂ activity.

### Example 7

### Once a Day Dosing of A-002

Up to 120 human subjects with stable CAD will be randomized to receive either placebo or one of two dosages (500 mg or 250 mg) of A-002 via once a day oral administration over an eight week time period. Subjects that are receiving statins or other standard cardiovascular therapeutics at the outset of the trial will continue to receive those therapeutics throughout the trial. However, subjects will not be permitted to receive systemic corticosteroids or inhaled steroids, high dose antioxidants or omega 3 fatty acids, immunosuppressant therapy, or anti-tumor necrosis factor therapy (e.g., infliximab) during the trial.

sPLA₂ levels will be measured at the outset of the trial and at two, four, and eight weeks after the start of A-002 administration. Plasma A-002 concentrations will also be measured at various times throughout the trial. sPLA₂ levels at each timepoint will be compared to the baseline measurement from the start of the trial to determine the effects of A-002 administration. Subjects receiving A-002 treatment will exhibit a substantial decrease in sPLA₂ levels.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Boekholdt, S. M. et al. 2005. Arterioscler Thromb Vasc Biol 25:839.
2. Cunningham, T. J., et al. 2006. J Neuroinflamm 3:26.
3. Hurt-Camejo, E., et al. 2001. Circulation Res 89:298.
4. Jamal, O. S., et al. 1998. Ann Rheum Dis 57:550-558)
5. Moses, G. S. D., et al. 2006. J Neuroinflamm 3:28.

TABLE 1
sPLA₂ standards in buffer, purified serum, and normal serum at 30 minutes

| | Standards in buffer only | | | Standards in purified serum | | | Standards in normal serum | | |
|---|---|---|---|---|---|---|---|---|---|
| | (Abs₄₀₅) | | | (Abs₄₀₅) | | | (Abs₄₀₅) | | |
| [sPLA₂] | Sample 1 | Sample 2 | Mean | Sample 1 | Sample 2 | Mean | Sample 1 | Sample 2 | Mean |
| 4.00 ng/ml | 0.621 | 0.646 | 0.633 | 0.562 | 0.542 | 0.552 | 0.662 | 0.444 | 0.553 |
| 2.00 ng/ml | 0.469 | 0.431 | 0.450 | 0.429 | 0.414 | 0.422 | 0.644 | 0.653 | 0.649 |
| 1.00 ng/ml | 0.276 | 0.263 | 0.270 | 0.300 | 0.293 | 0.297 | 0.606 | 0.632 | 0.619 |
| 0.50 ng/ml | 0.160 | 0.156 | 0.158 | 0.213 | 0.210 | 0.211 | 0.592 | 0.608 | 0.600 |
| 0.25 ng/ml | 0.107 | 0.107 | 0.107 | 0.166 | 0.164 | 0.165 | 0.574 | 0.592 | 0.583 |
| 0.13 ng/ml | 0.086 | 0.084 | 0.085 | 0.145 | 0.144 | 0.145 | 0.602 | 0.610 | 0.606 |
| 0.06 ng/ml | 0.077 | 0.076 | 0.076 | 0.131 | 0.133 | 0.132 | 0.609 | 0.609 | 0.609 |
| 0 ng/ml | 0.069 | 0.068 | 0.068 | 0.121 | 0.126 | 0.123 | 0.575 | 0.592 | 0.584 |

TABLE 2
sPLA₂ standards in buffer, purified serum, and normal serum at 60 minutes

| | Standards in buffer only | | | Standards in purified serum | | | Standards in normal serum | | |
|---|---|---|---|---|---|---|---|---|---|
| | (Abs₄₀₅) | | | (Abs₄₀₅) | | | (Abs₄₀₅) | | |
| [sPLA₂] | Sample 1 | Sample 2 | Mean | Sample 1 | Sample 2 | Mean | Sample 1 | Sample 2 | Mean |
| 4.00 ng/ml | 0.808 | 0.835 | 0.821 | 0.736 | 0.707 | 0.721 | 0.858 | 0.407 | 0.632 |
| 2.00 ng/ml | 0.614 | 0.564 | 0.589 | 0.562 | 0.541 | 0.552 | 0.838 | 0.853 | 0.845 |
| 1.00 ng/ml | 0.354 | 0.338 | 0.346 | 0.388 | 0.378 | 0.383 | 0.788 | 0.829 | 0.809 |
| 0.50 ng/ml | 0.196 | 0.192 | 0.194 | 0.270 | 0.265 | 0.268 | 0.772 | 0.797 | 0.784 |
| 0.25 ng/ml | 0.124 | 0.124 | 0.124 | 0.204 | 0.202 | 0.203 | 0.749 | 0.775 | 0.762 |
| 0.13 ng/ml | 0.094 | 0.091 | 0.093 | 0.176 | 0.175 | 0.176 | 0.784 | 0.795 | 0.790 |
| 0.06 ng/ml | 0.081 | 0.080 | 0.081 | 0.157 | 0.159 | 0.158 | 0.793 | 0.794 | 0.794 |
| 0 ng/ml | 0.070 | 0.069 | 0.069 | 0.142 | 0.151 | 0.147 | 0.750 | 0.769 | 0.759 |

TABLE 3
sPLA₂ standards in buffer, purified serum, and normal serum at 90 minutes

| | Standards in buffer only | | | Standards in purified serum | | | Standards in normal serum | | |
|---|---|---|---|---|---|---|---|---|---|
| | (Abs₄₀₅) | | | (Abs₄₀₅) | | | (Abs₄₀₅) | | |
| [sPLA₂] | Sample 1 | Sample 2 | Mean | Sample 1 | Sample 2 | Mean | Sample 1 | Sample 2 | Mean |
| 4.00 ng/ml | 1.088 | 1.117 | 1.102 | 1.031 | 0.998 | 1.014 | 1.163 | 1.184 | 1.173 |
| 2.00 ng/ml | 0.866 | 0.810 | 0.838 | 0.817 | 0.792 | 0.804 | 1.141 | 1.159 | 1.150 |
| 1.00 ng/ml | 0.512 | 0.489 | 0.501 | 0.568 | 0.555 | 0.562 | 1.096 | 1.138 | 1.117 |
| 0.50 ng/ml | 0.268 | 0.262 | 0.265 | 0.388 | 0.382 | 0.385 | 1.079 | 1.106 | 1.093 |
| 0.25 ng/ml | 0.157 | 0.156 | 0.156 | 0.284 | 0.281 | 0.282 | 1.053 | 1.082 | 1.068 |
| 0.13 ng/ml | 0.109 | 0.105 | 0.107 | 0.240 | 0.239 | 0.239 | 1.097 | 1.107 | 1.102 |
| 0.06 ng/ml | 0.089 | 0.086 | 0.087 | 0.209 | 0.213 | 0.211 | 1.103 | 1.104 | 1.103 |
| 0 ng/ml | 0.071 | 0.069 | 0.070 | 0.183 | 0.197 | 0.190 | 1.041 | 1.060 | 1.050 |

TABLE 4
sPLA₂ standards in buffer, purified serum, and normal serum at 120 minutes

| | Standards in buffer only | | | Standards in purified serum | | | Standards in normal serum | | |
|---|---|---|---|---|---|---|---|---|---|
| | (Abs₄₀₅) | | | (Abs₄₀₅) | | | (Abs₄₀₅) | | |
| [sPLA₂] | Sample 1 | Sample 2 | Mean | Sample 1 | Sample 2 | Mean | Sample 1 | Sample 2 | Mean |
| 4.00 ng/ml | 1.189 | 1.186 | 1.187 | 1.187 | 1.182 | 1.185 | 1.307 | 1.297 | 1.302 |
| 2.00 ng/ml | 1.043 | 1.016 | 1.029 | 1.040 | 1.025 | 1.033 | 1.302 | 1.297 | 1.299 |
| 1.00 ng/ml | 0.664 | 0.646 | 0.655 | 0.755 | 0.748 | 0.752 | 1.288 | 1.292 | 1.290 |
| 0.50 ng/ml | 0.344 | 0.339 | 0.341 | 0.519 | 0.515 | 0.517 | 1.282 | 1.284 | 1.283 |
| 0.25 ng/ml | 0.191 | 0.192 | 0.192 | 0.375 | 0.374 | 0.375 | 1.266 | 1.271 | 1.269 |
| 0.13 ng/ml | 0.126 | 0.121 | 0.123 | 0.313 | 0.313 | 0.313 | 1.305 | 1.300 | 1.302 |
| 0.06 ng/ml | 0.096 | 0.094 | 0.095 | 0.268 | 0.276 | 0.272 | 1.298 | 1.297 | 1.298 |
| 0 ng/ml | 0.072 | 0.070 | 0.071 | 0.232 | 0.251 | 0.241 | 1.226 | 1.229 | 1.228 |

TABLE 5
sPLA₂ standards normal serum, 10% purified serum, 1% purified serum, and 0.1%
purified serum at 60 minutes

| | Normal serum | | | 10% purified serum | | | 1% purified serum | | | 0.1% purified serum | | |
|---|---|---|---|---|---|---|---|---|---|---|---|---|
| | (Abs₄₀₅) | | | (Abs₄₀₅) | | | (Abs₄₀₅) | | | (Abs₄₀₅) | | |
| [sPLA₂] | Mean | SD | % CV | Mean | SD | % CV | Mean | SD | % CV | Mean | SD | % CV |
| 4.00 ng/ml | 0.749 | 0.014 | 1.9 | 0.786 | 0.017 | 2.1 | 0.817 | 0.033 | 4.0 | 0.798 | 0.003 | 0.4 |
| 2.00 ng/ml | 0.575 | 0.021 | 3.6 | 0.568 | 0.016 | 2.8 | 0.582 | 0.005 | 0.8 | 0.571 | 0.009 | 1.6 |
| 1.00 ng/ml | 0.405 | 0.024 | 5.9 | 0.351 | 0.001 | 0.3 | 0.357 | 0.007 | 2.1 | 0.324 | 0.008 | 2.3 |
| 0.50 ng/ml | 0.285 | 0.007 | 2.6 | 0.208 | 0.002 | 0.7 | 0.206 | 0.001 | 0.3 | 0.191 | 0.003 | 1.5 |
| 0.25 ng/ml | 0.218 | 0.000 | 0.1 | 0.133 | 0.003 | 1.9 | 0.132 | 0.002 | 1.7 | 0.128 | 0.000 | 0.2 |
| 0.13 ng/ml | 0.189 | 0.002 | 1.1 | 0.105 | 0.001 | 0.8 | 0.103 | 0.002 | 1.5 | 0.098 | 0.001 | 1.3 |
| 0.06 ng/ml | 0.172 | 0.001 | 0.3 | 0.089 | 0.001 | 0.6 | 0.089 | 0.002 | 2.7 | 0.083 | 0.001 | 1.0 |
| 0 ng/ml | 0.148 | 0.000 | 0.3 | 0.072 | 0.002 | 2.7 | 0.071 | 0.001 | 1.9 | 0.069 | 0.001 | 1.5 |

TABLE 6
sPLA₂ standards normal serum, 10% purified serum, 1% purified serum, and 0.1%
purified serum at 90 minutes

| | Normal serum | | | 10% purified serum | | | 1% purified serum | | | 0.1% purified serum | | |
|---|---|---|---|---|---|---|---|---|---|---|---|---|
| | (Abs₄₀₅) | | | (Abs₄₀₅) | | | (Abs₄₀₅) | | | (Abs₄₀₅) | | |
| [sPLA₂] | Mean | SD | % CV | Mean | SD | % CV | Mean | SD | % CV | Mean | SD | % CV |
| 4.00 ng/ml | 1.000 | 0.019 | 1.9 | 1.039 | 0.018 | 1.8 | 1.075 | 0.038 | 3.5 | 1.055 | 0.009 | 0.9 |
| 2.00 ng/ml | 0.787 | 0.027 | 3.5 | 0.774 | 0.022 | 2.9 | 0.794 | 0.009 | 1.2 | 0.780 | 0.010 | 1.3 |
| 1.00 ng/ml | 0.553 | 0.033 | 5.9 | 0.476 | 0.003 | 0.5 | 0.485 | 0.010 | 2.0 | 0.439 | 0.012 | 2.7 |
| 0.50 ng/ml | 0.383 | 0.010 | 2.6 | 0.272 | 0.002 | 0.8 | 0.268 | 0.002 | 0.9 | 0.250 | 0.003 | 1.1 |
| 0.25 ng/ml | 0.286 | 0.001 | 0.2 | 0.163 | 0.004 | 2.4 | 0.163 | 0.003 | 1.6 | 0.157 | 0.000 | 0.0 |
| 0.13 ng/ml | 0.242 | 0.001 | 0.5 | 0.122 | 0.000 | 0.0 | 0.119 | 0.002 | 2.1 | 0.113 | 0.002 | 1.9 |
| 0.06 ng/ml | 0.219 | 0.000 | 0.2 | 0.098 | 0.001 | 0.8 | 0.098 | 0.004 | 3.8 | 0.090 | 0.000 | 0.5 |
| 0 ng/ml | 0.184 | 0.001 | 0.3 | 0.074 | 0.002 | 3.0 | 0.072 | 0.001 | 2.0 | 0.069 | 0.001 | 2.0 |

TABLE 7
sPLA₂ standards and quality control (QC) samples in normal serum, 10% purified serum, 1% purified
serum, and 0.1% purified serum at 60 minutes

| | Purified serum | | | | | 10% purified | | 1% purified | | 0.1% purified | |
|---|---|---|---|---|---|---|---|---|---|---|---|
| | (Abs₄₀₅) | | | | | serum (Abs₄₀₅) | | serum (Abs₄₀₅) | | serum (Abs₄₀₅) | |
| [sPLA₂] | Sample 1 | Sample 2 | Mean | SD | % CV | Sample 1 | Sample 2 | Sample 1 | Sample 2 | Sample 1 | Sample 2 |
| 6.00 ng/ml | 0.894 | 0.870 | 0.882 | 0.017 | 1.9 | 0.829 | 0.872 | 0.829 | 0.859 | 0.841 | 0.848 |
| 4.00 ng/ml | 0.806 | 0.737 | 0.772 | 0.049 | 6.3 | 0.788 | 0.797 | 0.760 | 0.779 | 0.760 | 0.691 |
| 2.00 ng/ml | 0.645 | 0.598 | 0.621 | 0.034 | 5.4 | 0.584 | 0.659 | 0.606 | 0.602 | 0.626 | 0.610 |
| 2.00 ng/ml | 0.553 | 0.605 | 0.579 | 0.037 | 6.4 | 0.569 | 0.584 | 0.569 | 0.559 | 0.555 | 0.555 |
| (QC) | | | | | | | | | | | |
| 1.00 ng/ml | 0.409 | 0.416 | 0.412 | 0.005 | 1.2 | 0.371 | 0.391 | 0.409 | 0.365 | 0.336 | 0.357 |
| 1.00 ng/ml | 0.407 | 0.391 | 0.399 | 0.011 | 2.7 | 0.335 | 0.377 | 0.336 | 0.355 | 0.340 | 0.307 |
| (QC) | | | | | | | | | | | |
| 0.50 ng/ml | 0.289 | 0.284 | 0.287 | 0.003 | 1.2 | 0.253 | 0.261 | 0.234 | 0.260 | 0.246 | 0.234 |
| 0.50 ng/ml | 0.281 | 0.276 | 0.278 | 0.004 | 1.3 | 0.218 | 0.231 | 0.202 | 0.207 | 0.194 | 0.194 |
| (QC) | | | | | | | | | | | |
| 0.25 ng/ml | 0.193 | 0.204 | 0.198 | 0.008 | 3.9 | 0.131 | 0.134 | 0.161 | 0.138 | 0.116 | 0.122 |
| 0.06 ng/ml | 0.162 | 0.152 | 0.157 | 0.007 | 4.5 | 0.088 | 0.093 | 0.088 | 0.089 | 0.088 | 0.088 |
| 0 ng/ml | 0.143 | 0.140 | 0.141 | 0.002 | 1.6 | 0.073 | 0.073 | 0.070 | 0.070 | 0.071 | 0.069 |

TABLE 8
sPLA₂ standards and quality control (QC) samples in normal serum, 10% purified serum, 1% purified
serum, and 0.1% purified serum at 90 minutes

| | Purified serum | | | | | 10% purified | | 1% purified | | 0.1% purified | |
|---|---|---|---|---|---|---|---|---|---|---|---|
| | (Abs₄₀₅) | | | | | serum (Abs₄₀₅) | | serum (Abs₄₀₅) | | serum (Abs₄₀₅) | |
| [sPLA₂] | Sample 1 | Sample 2 | Mean | SD | % CV | Sample 1 | Sample 2 | Sample 1 | Sample 2 | Sample 1 | Sample 2 |
| 6.00 ng/ml | 1.148 | 1.124 | 1.136 | 0.017 | 1.5 | 1.086 | 1.132 | 1.080 | 1.116 | 1.093 | 1.096 |
| 4.00 ng/ml | 1.053 | 0.978 | 1.016 | 0.054 | 5.3 | 1.037 | 1.049 | 1.004 | 1.027 | 1.003 | 0.926 |
| 2.00 ng/ml | 0.870 | 0.804 | 0.837 | 0.047 | 5.6 | 0.795 | 0.889 | 0.820 | 0.815 | 0.847 | 0.827 |
| 2.00 ng/ml | 0.752 | 0.816 | 0.784 | 0.046 | 5.9 | 0.775 | 0.797 | 0.770 | 0.760 | 0.759 | 0.751 |
| (QC) | | | | | | | | | | | |
| 1.00 ng/ml | 0.554 | 0.564 | 0.559 | 0.007 | 1.3 | 0.508 | 0.533 | 0.558 | 0.498 | 0.455 | 0.487 |
| 1.00 ng/ml | 0.556 | 0.536 | 0.546 | 0.014 | 2.6 | 0.453 | 0.514 | 0.460 | 0.483 | 0.464 | 0.412 |
| (QC) | | | | | | | | | | | |
| 0.50 ng/ml | 0.388 | 0.375 | 0.381 | 0.009 | 2.4 | 0.337 | 0.347 | 0.309 | 0.349 | 0.326 | 0.311 |
| 0.50 ng/ml | 0.375 | 0.372 | 0.373 | 0.003 | 0.7 | 0.287 | 0.306 | 0.265 | 0.271 | 0.253 | 0.251 |
| (QC) | | | | | | | | | | | |
| 0.25 ng/ml | 0.250 | 0.261 | 0.256 | 0.008 | 3.1 | 0.160 | 0.162 | 0.158 | 0.171 | 0.138 | 0.147 |
| 0.06 ng/ml | 0.205 | 0.188 | 0.197 | 0.012 | 6.0 | 0.097 | 0.104 | 0.096 | 0.098 | 0.096 | 0.096 |
| 0 ng/ml | 0.176 | 0.171 | 0.174 | 0.004 | 2.3 | 0.075 | 0.075 | 0.071 | 0.072 | 0.072 | 0.070 |

TABLE 9
Determination of percent recovery for quality control samples using 60 minute purified
serum standard curve

| | Purified serum | | | | | | | |
|---|---|---|---|---|---|---|---|---|
| | (Abs₄₀₅) | | | | | | | |
| | | | | | % | Interpolated X | | |
| [sPLA₂] | Sample 1 | Sample 2 | Mean | SD | CV | means | Anti-log | % Recovery |
| 6.00 ng/ml | 0.894 | 0.870 | 0.882 | 0.017 | 1.9 | — | — | — |
| 4.00 ng/ml | 0.806 | 0.737 | 0.772 | 0.049 | 6.3 | — | — | — |
| 2.00 ng/ml | 0.645 | 0.598 | 0.621 | 0.034 | 5.4 | — | — | — |
| 2.00 ng/ml | 0.553 | 0.605 | 0.579 | 0.037 | 6.4 | 0.261 | 1.82 | 91 |
| (QC) | | | | | | | | |
| 1.00 ng/ml | 0.409 | 0.416 | 0.412 | 0.005 | 1.2 | — | — | — |
| 1.00 ng/ml | 0.407 | 0.391 | 0.399 | 0.011 | 2.7 | −0.042 | 0.91 | 91 |
| (QC) | | | | | | | | |
| 0.50 ng/ml | 0.289 | 0.284 | 0.287 | 0.003 | 1.2 | — | — | — |
| 0.50 ng/ml | 0.281 | 0.276 | 0.278 | 0.004 | 1.3 | −0.333 | 0.46 | 93 |
| (QC) | | | | | | | | |
| 0.25 ng/ml | 0.193 | 0.204 | 0.198 | 0.008 | 3.9 | — | — | — |
| 0.06 ng/ml | 0.162 | 0.152 | 0.157 | 0.007 | 4.5 | — | — | — |
| 0 ng/ml | 0.143 | 0.140 | 0.141 | 0.002 | 1.6 | — | — | — |

TABLE 10
Determination of percent recovery for quality control samples using 90 minute
purified serum standard curve

| | Purified serum | | | | | | | |
|---|---|---|---|---|---|---|---|---|
| | (Abs₄₀₅) | | | | | Interpolated X | | |
| [sPLA₂] | Sample 1 | Sample 2 | Mean | SD | % CV | means | Anti-log | % Recovery |
| 6.00 ng/ml | 1.148 | 1.124 | 1.136 | 0.017 | 1.5 | — | — | — |
| 4.00 ng/ml | 1.053 | 0.978 | 1.016 | 0.054 | 5.3 | — | — | — |
| 2.00 ng/ml | 0.870 | 0.804 | 0.837 | 0.047 | 5.6 | — | — | — |
| 2.00 ng/ml | 0.752 | 0.816 | 0.784 | 0.046 | 5.9 | 0.261 | 1.82 | 91 |
| (QC) | | | | | | | | |
| 1.00 ng/ml | 0.554 | 0.564 | 0.559 | 0.007 | 1.3 | — | — | — |
| 1.00 ng/ml | 0.556 | 0.536 | 0.546 | 0.014 | 2.6 | −0.034 | 0.92 | 92 |
| (QC) | | | | | | | | |
| 0.50 ng/ml | 0.388 | 0.375 | 0.381 | 0.009 | 2.4 | — | — | — |
| 0.50 ng/ml | 0.375 | 0.372 | 0.373 | 0.003 | 0.7 | −0.321 | 0.48 | 96 |
| (QC) | | | | | | | | |
| 0.25 ng/ml | 0.250 | 0.261 | 0.256 | 0.008 | 3.1 | — | — | — |
| 0.06 ng/ml | 0.205 | 0.188 | 0.197 | 0.012 | 6.0 | — | — | — |
| 0 ng/ml | 0.176 | 0.171 | 0.174 | 0.004 | 2.3 | — | — | — |

TABLE 11
Determination of percent recovery for quality control samples using 60
minute purified serum standard curve

| | Purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 0.903 | 0.908 | 0.906 | 0.004 | 0.4 | — | — | — |
| 4.00 ng/ml | 0.777 | 0.797 | 0.787 | 0.014 | 1.8 | — | — | — |
| 2.00 ng/ml | 0.644 | 0.632 | 0.638 | 0.008 | 1.3 | — | — | — |
| 2.00 ng/ml | 0.571 | 0.603 | 0.587 | 0.023 | 3.9 | 0.235 | 1.72 | 86 |
| (QC) | | | | | | | | |
| 1.00 ng/ml | 0.456 | 0.457 | 0.456 | 0.001 | 0.3 | — | — | — |
| 1.00 ng/ml | 0.388 | 0.426 | 0.407 | 0.027 | 6.5 | −0.125 | 0.75 | 75 |
| (QC) | | | | | | | | |
| 0.50 ng/ml | 0.342 | 0.348 | 0.345 | 0.004 | 1.3 | — | — | — |
| 0.50 ng/ml | 0.310 | 0.311 | 0.311 | 0.001 | 0.3 | −0.406 | 0.39 | 79 |
| (QC) | | | | | | | | |
| 0.25 ng/ml | 0.228 | 0.234 | 0.231 | 0.004 | 1.6 | — | — | — |
| 0.06 ng/ml | 0.196 | 0.201 | 0.198 | 0.003 | 1.6 | — | — | — |
| 0 ng/ml | 0.171 | 0.174 | 0.173 | 0.002 | 1.2 | — | — | — |

TABLE 12
Determination of percent recovery for quality control samples using 60 minute
10% purified serum standard curve

| | 10% purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 0.898 | 0.944 | 0.921 | 0.033 | 3.5 | — | — | — |
| 4.00 ng/ml | 0.850 | 0.870 | 0.860 | 0.015 | 1.7 | — | — | — |
| 2.00 ng/ml | 0.661 | 0.657 | 0.659 | 0.003 | 0.4 | — | — | — |
| 2.00 ng/ml | 0.678 | 0.675 | 0.677 | 0.002 | 0.3 | 0.322 | 2.10 | 105 |
| (QC) | | | | | | | | |
| 1.00 ng/ml | 0.415 | 0.417 | 0.416 | 0.001 | 0.3 | — | — | — |
| 1.00 ng/ml | 0.427 | 0.426 | 0.426 | 0.000 | 0.1 | 0.012 | 1.03 | 103 |
| (QC) | | | | | | | | |
| 0.50 ng/ml | 0.234 | 0.236 | 0.235 | 0.001 | 0.4 | — | — | — |
| 0.50 ng/ml | 0.259 | 0.261 | 0.260 | 0.001 | 0.5 | −0.247 | 0.57 | 113 |
| (QC) | | | | | | | | |
| 0.25 ng/ml | 0.124 | 0.122 | 0.123 | 0.001 | 0.7 | — | — | — |
| 0.06 ng/ml | 0.088 | 0.088 | 0.088 | 0.000 | 0.1 | — | — | — |
| 0 ng/ml | 0.072 | 0.072 | 0.072 | 0.000 | 0.2 | — | — | — |

TABLE 13
Determination of percent recovery for quality control samples using 60 minute
1% purified serum standard curve

| | 1% purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 0.963 | 0.947 | 0.955 | 0.011 | 1.2 | — | — | — |
| 4.00 ng/ml | 0.870 | 0.862 | 0.866 | 0.006 | 0.7 | — | — | — |
| 2.00 ng/ml | 0.678 | 0.673 | 0.676 | 0.004 | 0.5 | — | — | — |
| 2.00 ng/ml | 0.698 | 0.680 | 0.689 | 0.012 | 1.8 | 0.325 | 2.11 | 106 |
| (QC) | | | | | | | | |
| 1.00 ng/ml | 0.426 | 0.429 | 0.428 | 0.002 | 0.4 | — | — | — |
| 1.00 ng/ml | 0.482 | 0.431 | 0.457 | 0.036 | 7.8 | 0.036 | 1.09 | 109 |
| (QC) | | | | | | | | |
| 0.50 ng/ml | 0.240 | 0.244 | 0.242 | 0.003 | 1.2 | — | — | — |
| 0.50 ng/ml | 0.241 | 0.243 | 0.242 | 0.002 | 0.8 | −0.308 | 0.49 | 98 |
| (QC) | | | | | | | | |
| 0.25 ng/ml | 0.120 | 0.129 | 0.125 | 0.006 | 4.9 | — | — | — |
| 0.06 ng/ml | 0.081 | 0.082 | 0.082 | 0.001 | 0.9 | — | — | — |
| 0 ng/ml | 0.065 | 0.068 | 0.067 | 0.002 | 2.3 | — | — | — |

TABLE 14
Determination of percent recovery for quality control samples using 60 minute
0.1% purified serum standard curve

| | 0.1% purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 0.967 | 0.941 | 0.954 | 0.018 | 1.9 | — | — | — |
| 4.00 ng/ml | 0.876 | 0.873 | 0.875 | 0.002 | 0.2 | — | — | — |
| 2.00 ng/ml | 0.680 | 0.684 | 0.682 | 0.003 | 0.5 | — | — | — |
| 2.00 ng/ml | 0.686 | 0.685 | 0.686 | 0.001 | 0.1 | 0.312 | 2.05 | 102 |
| (QC) | | | | | | | | |
| 1.00 ng/ml | 0.431 | 0.440 | 0.435 | 0.007 | 1.5 | — | — | — |
| 1.00 ng/ml | 0.420 | 0.408 | 0.414 | 0.008 | 2.0 | −0.037 | 0.92 | 92 |
| (QC) | | | | | | | | |
| 0.50 ng/ml | 0.255 | 0.262 | 0.258 | 0.005 | 2.1 | — | — | — |
| 0.50 ng/ml | 0.247 | 0.245 | 0.246 | 0.002 | 0.6 | −0.324 | 0.47 | 95 |
| (QC) | | | | | | | | |
| 0.25 ng/ml | 0.128 | 0.125 | 0.126 | 0.002 | 1.6 | — | — | — |
| 0.06 ng/ml | 0.083 | 0.084 | 0.084 | 0.001 | 0.6 | — | — | — |
| 0 ng/ml | 0.066 | 0.067 | 0.067 | 0.000 | 0.6 | — | — | — |

TABLE 15
Summary of quality control results using normal serum, 10% purified serum,
1% purified serum, and 0.1% purified serum 60 minute standard curves

| Quality control | Nominal sPLA₂ | Nominal sPLA₂ | Nominal sPLA₂ | Nominal sPLA₂ |
|---|---|---|---|---|
| sample sPLA₂ | concentration in | concentration in 10% | concentration in 1% | concentration in |
| concentration | purified serum | purified serum | purified serum | 0.1% purified serum |
| (ng/ml) | (ng/ml) | (ng/ml) | (ng/ml) | (ng/ml) |
| 2.00 | 1.72 | 2.10 | 2.11 | 2.05 |
| 1.00 | 0.75 | 1.03 | 1.09 | 0.92 |
| 0.50 | 0.39 | 0.57 | 0.49 | 0.47 |

TABLE 16
Determination of percent recovery for quality control samples using 90 minute
purified serum standard curve

| | Purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 1.085 | 1.087 | 1.086 | 0.001 | 0.1 | — | — | — |
| 4.00 ng/ml | 0.956 | 0.975 | 0.966 | 0.013 | 1.4 | — | — | — |
| 2.00 ng/ml | 0.807 | 0.789 | 0.798 | 0.013 | 1.6 | — | — | — |
| 2.00 ng/ml | 0.724 | 0.751 | 0.738 | 0.019 | 2.6 | 0.233 | 1.71 | 86 |
| (QC) | | | | | | | | |
| 1.00 ng/ml | 0.575 | 0.577 | 0.576 | 0.001 | 0.2 | — | — | — |
| 1.00 ng/ml | 0.489 | 0.551 | 0.520 | 0.044 | 8.4 | −0.112 | 0.77 | 77 |
| (QC) | | | | | | | | |
| 0.50 ng/ml | 0.429 | 0.436 | 0.433 | 0.005 | 1.1 | — | — | — |
| 0.50 ng/ml | 0.387 | 0.370 | 0.379 | 0.012 | 3.2 | −0.427 | 0.37 | 75 |
| (QC) | | | | | | | | |
| 0.25 ng/ml | 0.280 | 0.289 | 0.285 | 0.006 | 2.2 | — | — | — |
| 0.06 ng/ml | 0.238 | 0.241 | 0.240 | 0.002 | 0.9 | — | — | — |
| 0 ng/ml | 0.203 | 0.207 | 0.205 | 0.003 | 1.4 | — | — | — |

TABLE 16
Determination of percent recovery for quality control samples using 90 minute
10% purified serum standard curve

| | 10% purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 1.081 | 1.125 | 1.103 | 0.031 | 2.8 | — | — | — |
| 4.00 ng/ml | 1.036 | 1.053 | 1.045 | 0.012 | 1.2 | — | — | — |
| 2.00 ng/ml | 0.826 | 0.822 | 0.824 | 0.003 | 0.3 | — | — | — |
| 2.00 ng/ml | 0.798 | 0.807 | 0.803 | 0.006 | 0.8 | 0.278 | 1.90 | 95 |
| (QC) | | | | | | | | |
| 1.00 ng/ml | 0.521 | 0.525 | 0.523 | 0.003 | 0.5 | — | — | — |
| 1.00 ng/ml | 0.508 | 0.502 | 0.505 | 0.004 | 0.8 | −0.023 | 0.95 | 95 |
| (QC) | | | | | | | | |
| 0.50 ng/ml | 0.288 | 0.291 | 0.290 | 0.002 | 0.7 | — | — | — |
| 0.50 ng/ml | 0.303 | 0.288 | 0.296 | 0.011 | 3.6 | −0.288 | 0.51 | 103 |
| (QC) | | | | | | | | |
| 0.25 ng/ml | 0.141 | 0.141 | 0.141 | 0.000 | 0.0 | — | — | — |
| 0.06 ng/ml | 0.095 | 0.095 | 0.095 | 0.000 | 0.0 | — | — | — |
| 0 ng/ml | 0.073 | 0.074 | 0.074 | 0.001 | 1.0 | — | — | — |

TABLE 17
Determination of percent recovery for quality control samples using 90 minute
1% purified serum standard curve

| | 1% purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 1.159 | 1.138 | 1.149 | 0.015 | 1.3 | — | — | — |
| 4.00 ng/ml | 1.063 | 1.049 | 1.056 | 0.010 | 0.9 | — | — | — |
| 2.00 ng/ml | 0.858 | 0.845 | 0.852 | 0.009 | 1.1 | — | — | — |
| 2.00 ng/ml | 0.876 | 0.851 | 0.864 | 0.018 | 2.0 | 0.323 | 2.11 | 105 |
| (QC) | | | | | | | | |
| 1.00 ng/ml | 0.543 | 0.542 | 0.543 | 0.001 | 0.1 | — | — | — |
| 1.00 ng/ml | 0.596 | 0.533 | 0.565 | 0.045 | 7.9 | 0.018 | 1.04 | 104 |
| (QC) | | | | | | | | |
| 0.50 ng/ml | 0.303 | 0.302 | 0.303 | 0.001 | 0.2 | — | — | — |
| 0.50 ng/ml | 0.295 | 0.272 | 0.284 | 0.016 | 5.7 | −0.334 | 0.46 | 93 |
| (QC) | | | | | | | | |
| 0.25 ng/ml | 0.138 | 0.140 | 0.139 | 0.001 | 1.0 | — | — | — |
| 0.06 ng/ml | 0.087 | 0.088 | 0.088 | 0.001 | 0.8 | — | — | — |
| 0 ng/ml | 0.065 | 0.067 | 0.066 | 0.001 | 2.1 | — | — | — |

TABLE 18
Determination of percent recovery for quality control samples using 90 minute
0.1% purified serum standard curve

| | 0.1% purified serum | | | | | | | |
|---|---|---|---|---|---|---|---|---|
| | (Abs₄₀₅) | | | | | Interpolated | | |
| [sPLA₂] | Sample 1 | Sample 2 | Mean | SD | % CV | X means | Anti-log | % Recovery |
| 6.00 ng/ml | 1.155 | 1.128 | 1.142 | 0.019 | 1.7 | — | — | — |
| 4.00 ng/ml | 1.065 | 1.062 | 1.064 | 0.002 | 0.2 | — | — | — |
| 2.00 ng/ml | 0.849 | 0.856 | 0.853 | 0.005 | 0.6 | — | — | — |
| 2.00 ng/ml | 0.755 | 0.751 | 0.753 | 0.003 | 0.4 | 0.202 | 1.59 | 80 |
| (QC) | | | | | | | | |
| 1.00 ng/ml | 0.543 | 0.553 | 0.548 | 0.007 | 1.3 | — | — | — |
| 1.00 ng/ml | 0.444 | 0.424 | 0.434 | 0.014 | 3.3 | −0.137 | 0.73 | 73 |
| (QC) | | | | | | | | |
| 0.50 ng/ml | 0.315 | 0.322 | 0.319 | 0.005 | 1.6 | — | — | — |
| 0.50 ng/ml | 0.252 | 0.244 | 0.248 | 0.006 | 2.3 | −0.420 | 0.38 | 76 |
| (QC) | | | | | | | | |
| 0.25 ng/ml | 0.146 | 0.136 | 0.141 | 0.007 | 5.0 | — | — | — |
| 0.06 ng/ml | 0.088 | 0.086 | 0.087 | 0.001 | 1.6 | — | — | — |
| 0 ng/ml | 0.065 | 0.065 | 0.065 | 0.000 | 0.0 | — | — | — |

TABLE 19
Summary of quality control results using normal serum, 10% purified serum,
1% purified serum, and 0.1% purified serum 90 minute standard curves

| Quality control | Nominal sPLA₂ | Nominal sPLA₂ | Nominal sPLA₂ | Nominal sPLA₂ |
|---|---|---|---|---|
| sample sPLA₂ | concentration in | concentration in 10% | concentration in 1% | concentration in |
| concentration | purified serum | purified serum | purified serum | 0.1% purified serum |
| (ng/ml) | (ng/ml) | (ng/ml) | (ng/ml) | (ng/ml) |
| 2.00 | 1.71 | 1.90 | 2.11 | 1.59 |
| 1.00 | 0.77 | 0.95 | 1.04 | 0.73 |
| 0.50 | 0.37 | 0.51 | 0.46 | 0.38 |

TABLE 20
Average quality control results for 60 and 90 minute development times using normal
serum, 10% purified serum, 1% purified serum, and 0.1% purified serum standard curves

| Quality control | Nominal sPLA₂ | Nominal sPLA₂ | Nominal sPLA₂ | Nominal sPLA₂ |
|---|---|---|---|---|
| sample sPLA₂ | concentration in | concentration in 10% | concentration in 1% | concentration in |
| concentration | purified serum | purified serum | purified serum | 0.1% purified serum |
| (ng/ml) | (ng/ml) | (ng/ml) | (ng/ml) | (ng/ml) |
| 2.00 | 1.72 | 2.00 | 2.11 | 1.82 |
| 1.00 | 0.76 | 0.99 | 1.06 | 0.82 |
| 0.50 | 0.38 | 0.54 | 0.48 | 0.43 |

TABLE 21
Changes in sPLA2 levels in ITT population

| | | A-002 | A-002 | A-002 | A-002 | A-002 | |
|---|---|---|---|---|---|---|---|
| | | (50 mg) | (100 mg) | (250 mg) | (500 mg) | Total | Placebo |
| Baseline | # of | 16 | 16 | 15 | 17 | 64 | 16 |
| | subjects | | | | | | |
| | Mean (SD) | 3.24 ng/ml | 2.86 ng/ml | 2.88 ng/ml | 2.96 ng/ml | 2.99 ng/ml | 2.97 ng/ml |
| | | (2.05) | (1.52) | (1.35) | (1.31) | (1.56) | (1.60) |
| | Median | 2.65 ng/ml | 2.95 ng/ml | 2.40 ng/ml | 2.80 ng/ml | 2.70 ng/ml | 2.40 ng/ml |
| | Range | 0.80 to 8.80 | 0.05 to 6.00 | 1.30 to 5.20 | 0.60 to 5.40 | 0.05 to 8.80 | 0.80 to 5.70 |
| Week 2 | # of | 12 | 14 | 14 | 15 | 55 | 17 |
| | subjects | | | | | | |
| | Mean (SD) | 0.89 ng/ml | 0.68 ng/ml | 0.73 ng/ml | 0.18 ng/ml | 0.60 ng/ml | 3.11 ng/ml |
| | | (1.06) | (0.73) | (1.11) | (0.19) | (0.86) | (1.24) |
| | Median | 0.45 ng/ml | 0.45 ng/ml | 0.35 ng/ml | 0.05 ng/ml | 0.30 ng/ml | 2.80 ng/ml |
| | Range | 0.20 to 4.00 | 0.05 to 2.90 | 0.05 to 4.40 | 0.05 to 0.60 | 0.05 to 4.40 | 1.40 to 5.70 |
| Change from | # of | 12 | 14 | 13 | 15 | 54 | 16 |
| baseline at week 2 | subjects | | | | | | |
| | observed | | | | | | |
| | Mean (SD) | −2.74 ng/ml | −1.81 ng/ml | −2.02 ng/ml | −2.84 ng/ml | −2.35 ng/ml | +0.02 ng/ml |
| | | (1.60) | (1.62) | (1.20) | (1.26) | (1.46) | (1.31) |
| | Median | −2.60 ng/ml | −1.85 ng/ml | −1.90 ng/ml | −2.95 ng/ml | −2.40 ng/ml | +0.10 ng/ml |
| | Range | −5.30 to −0.60 | −3.70 to +2.85 | −4.20 to −0.20 | −4.80 to −0.55 | −5.30 to +2.85 | −1.80 to +3.90 |
| | p-value of | 0.0001 | 0.0067 | <0.0001 | <0.0001 | <0.0001 | 0.9451 |
| | change | | | | | | |
| | within | | | | | | |
| | group | | | | | | |
| Week 4 | # of | 13 | 14 | 14 | 15 | 56 | 15 |
| | subjects | | | | | | |
| | observed | | | | | | |
| | Mean (SD) | 0.85 ng/ml | 0.49 ng/ml | 0.61 ng/ml | 0.15 ng/ml | 0.51 ng/ml | 4.39 ng/ml |
| | | (0.61) | (0.36) | (0.65) | (0.17) | (0.53) | (3.62) |
| | Median | 0.70 ng/ml | 0.50 ng/ml | 0.35 ng/ml | 0.05 ng/ml | 0.35 ng/ml | 2.90 ng/ml |
| | Range | 0.10 to 2.40 | 0.05 to 1.10 | 0.05 to 2.30 | 0.05 to 0.60 | 0.05 to 2.40 | 1.30 to 15.90 |
| Change from | # of | 12 | 13 | 13 | 15 | 53 | 15 |
| baseline at week 4 | subjects | | | | | | |
| | observed | | | | | | |
| | Mean (SD) | −2.50 ng/ml | −2.55 ng/ml | −2.13 ng/ml | −2.87 ng/ml | −2.53 ng/ml | +1.29 ng/ml |
| | | (1.77) | (1.51) | (1.19) | (1.30) | (1.43) | (3.11) |
| | Median | −2.15 ng/ml | −2.50 ng/ml | −2.20 ng/ml | −2.95 ng/ml | −2.35 ng/ml | +0.30 ng/ml |
| | Range | −6.40 to −0.60 | −5.70 to 0.00 | −4.20 to 0.20 | −4.80 to −0.55 | −6.40 to 0.20 | −1.20 to |
| | | | | | | | +10.20 |
| | p-value of | 0.0005 | <0.0001 | <0.0001 | <0.0001 | <0.0001 | 0.4129 |
| | change | | | | | | |
| | within | | | | | | |
| | group | | | | | | |

TABLE 22
Changes in sPLA2 levels in diabetes subgroup of ITT population

| | | A-002 | A-002 | A-002 | A-002 | A-002 | |
|---|---|---|---|---|---|---|---|
| | | (50 mg) | (100 mg) | (250 mg) | (500 mg) | Total | Placebo |
| Baseline | # of | 6 | 6 | 4 | 4 | 20 | 3 |
| | subjects | | | | | | |
| | Mean (SD) | 2.45 ng/ml | 2.78 ng/ml | 3.00 ng/ml | 3.73 ng/ml | 2.91 ng/ml | 4.30 ng/ml |
| | | (1.06) | (1.94) | (1.96) | (1.21) | (1.53) | (2.01) |
| | Median | 2.65 ng/ml | 2.50 ng/ml | 3.00 ng/ml | 3.40 ng/ml | 2.75 ng/ml | 5.20 ng/ml |
| | Range | 0.80 to 3.80 | 0.05 to 6.00 | 1.30 to 4.70 | 2.70 to 5.40 | 0.05 to 6.00 | 2.00 to 5.70 |
| Week 2 | # of | 4 | 5 | 3 | 3 | 15 | 4 |
| | subjects | | | | | | |
| | Mean (SD) | 0.55 ng/ml | 1.02 ng/ml | 0.60 ng/ml | 0.32 ng/ml | 0.67 ng/ml | 4.23 ng/ml |
| | | (0.40) | (1.07) | (0.46) | (0.28) | (0.69) | (1.35) |
| | Median | 0.45 ng/ml | 0.50 ng/ml | 0.50 ng/ml | 0.30 ng/ml | 0.50 ng/ml | 4.20 ng/ml |
| | Range | 0.20 to 1.10 | 0.40 to 2.90 | 0.20 to 1.10 | 0.05 to 0.60 | 0.05 to 2.90 | 2.80 to 5.70 |
| Change from | # of | 4 | 5 | 3 | 3 | 15 | 3 |
| baseline at week 2 | subjects | | | | | | |
| | observed | | | | | | |
| | Mean (SD) | −2.05 ng/ml | −1.11 ng/ml | −1.83 ng/ml | −3.75 ng/ml | −2.03 ng/ml | −0.33 ng/ml |
| | | (0.97) | (2.27) | (2.10) | (0.95) | (1.84) | (1.33) |
| | Median | −2.45 ng/ml | −1.80 ng/ml | −1.10 ng/ml | −3.50 ng/ml | −2.40 ng/ml | 0.00 ng/ml |
| | Range | −2.70 to −0.60 | −3.00 to +2.85 | −4.20 to −0.20 | −4.80 to −2.95 | −4.80 to +2.85 | −1.80 to +0.80 |
| | p-value of | 0.1250 | 0.4375 | 0.2694 | 0.0207 | 0.0008 | 0.7069 |
| | change | | | | | | |
| | within | | | | | | |
| | group | | | | | | |
| Week 4 | # of | 5 | 6 | 3 | 3 | 17 | 3 |
| | subjects | | | | | | |
| | observed | | | | | | |
| | Mean (SD) | 0.66 ng/ml | 0.48 ng/ml | 0.68 ng/ml | 0.25 ng/ml | 0.53 ng/ml | 8.43 ng/ml |
| | | (0.37) | (0.36) | (0.74) | (0.30) | (0.43) | (6.47) |
| | Median | 0.60 ng/ml | 0.40 ng/ml | 0.50 ng/ml | 0.10 ng/ml | 0.50 ng/ml | 4.80 ng/ml |
| | Range | 0.20 to 1.20 | 0.05 to 1.10 | 0.05 to 1.50 | 0.05 to 0.60 | 0.05 to 1.50 | 4.60 to 15.90 |
| Change from | # of | 5 | 6 | 3 | 3 | 17 | 3 |
| baseline at week 4 | subjects | | | | | | |
| | observed | | | | | | |
| | Mean (SD) | −1.76 ng/ml | −2.30 ng/ml | −1.75 ng/ml | −3.82 ng/ml | −2.31 ng/ml | +4.13 ng/ml |
| | | (1.01) | (1.90) | (2.24) | (0.93) | (1.64) | (5.52) |
| | Median | −1.50 ng/ml | −1.80 ng/ml | −1.25 ng/ml | −3.70 ng/ml | −1.90 ng/ml | +2.80 ng/ml |
| | Range | −3.20 to −0.60 | −5.70 to 0.00 | −4.20 to +0.20 | −4.80 to −2.95 | −5.70 to +0.20 | −0.60 to |
| | | | | | | | +10.20 |
| | p-value of | 0.0177 | 0.0311 | 0.3090 | 0.0192 | <0.0001 | 0.3242 |
| | change | | | | | | |
| | within | | | | | | |
| | group | | | | | | |

TABLE 23
Changes in sPLA2 levels in non-diabetes subgroup of ITT population

| | | A-002 | A-002 | A-002 | A-002 | A-002 | |
|---|---|---|---|---|---|---|---|
| | | (50 mg) | (100 mg) | (250 mg) | (500 mg) | Total | Placebo |
| Baseline | # of | 10 | 10 | 11 | 13 | 44 | 13 |
| | subjects | | | | | | |
| | Mean (SD) | 3.72 ng/ml | 2.91 ng/ml | 2.84 ng/ml | 2.73 ng/ml | 3.02 ng/ml | 2.66 ng/ml |
| | | (2.39) | (1.32) | (1.18) | (1.30) | (1.58) | (1.41) |
| | Median | 3.20 ng/ml | 3.20 ng/ml | 2.40 ng/ml | 2.70 ng/ml | 2.60 ng/ml | 2.30 ng/ml |
| | Range | 1.30 to 8.80 | 1.00 to 4.90 | 1.50 to 5.20 | 0.60 to 4.60 | 0.60 to 8.80 | 0.80 to 5.40 |
| Week 2 | # of | 8 | 9 | 11 | 12 | 40 | 13 |
| | subjects | | | | | | |
| | Mean (SD) | 1.06 ng/ml | 0.48 ng/ml | 0.76 ng/ml | 0.15 ng/ml | 0.57 ng/ml | 2.76 ng/ml |
| | | (1.27) | (0.42) | (1.25) | (0.15) | (0.92) | (1.02) |
| | Median | 0.06 ng/ml | 0.30 ng/ml | 0.30 ng/ml | 0.05 ng/ml | 0.30 ng/ml | 2.70 ng/ml |
| | Range | 0.20 to 4.00 | 0.05 to 1.20 | 0.05 to 4.40 | 0.05 to 0.50 | 0.05 to 4.40 | 1.40 to 4.70 |
| Change from | # of | 8 | 9 | 10 | 12 | 39 | 13 |
| baseline at week 2 | subjects | | | | | | |
| | observed | | | | | | |
| | Mean (SD) | −3.09 ng/ml | −2.21 ng/ml | −2.08 ng/ml | −2.61 ng/ml | −2.48 ng/ml | +0.10 ng/ml |
| | | (1.80) | (1.09) | (0.97) | (1.26) | (1.29) | (1.35) |
| | Median | −3.05 ng/ml | −2.40 ng/ml | −2.00 ng/ml | −2.68 ng/ml | −2.40 ng/ml | +0.20 ng/ml |
| | Range | −5.30 to −0.70 | −3.70 to −0.40 | −3.80 to −0.80 | −4.40 to −0.55 | −5.30 to −0.40 | −1.70 to +3.90 |
| | p-value of | 0.0018 | 0.0003 | <0.0001 | <0.0001 | <0.0001 | 0.9597 |
| | change | | | | | | |
| | within | | | | | | |
| | group | | | | | | |
| Week 4 | # of | 8 | 8 | 11 | 12 | 39 | 12 |
| | subjects | | | | | | |
| | observed | | | | | | |
| | Mean (SD) | 0.96 ng/ml | 0.51 ng/ml | 0.60 ng/ml | 0.13 ng/ml | 0.51 ng/ml | 3.38 ng/ml |
| | | (0.72) | (0.38) | (0.66) | (0.13) | (0.58) | (1.87) |
| | Median | 0.75 ng/ml | 0.60 ng/ml | 0.30 ng/ml | 0.05 ng/ml | 0.30 ng/ml | 2.65 ng/ml |
| | Range | 0.10 to 2.40 | 0.05 to 1.00 | 0.05 to 2.30 | 0.05 to 0.40 | 0.05 to 2.40 | 1.30 to 7.00 |
| Change from | # of | 7 | 7 | 10 | 12 | 36 | 12 |
| baseline at week 4 | subjects | | | | | | |
| | observed | | | | | | |
| | Mean (SD) | −3.03 ng/ml | −2.76 ng/ml | −2.25 ng/ml | −2.63 ng/ml | −2.63 ng/ml | +0.58 ng/ml |
| | | (2.07) | (1.20) | (0.83) | (1.30) | (1.33) | (2.00) |
| | Median | −2.80 ng/ml | −2.60 ng/ml | −2.20 ng/ml | −2.53 ng/ml | −2.45 ng/ml | +0.25 ng/ml |
| | Range | −6.40 to −0.80 | −4.40 to −0.90 | −3.50 to −1.20 | −4.35 to −0.55 | −6.40 to −0.55 | −1.20 to +6.20 |
| | p-value of | 0.0082 | 0.0009 | <0.0001 | <0.0001 | <0.0001 | 0.7334 |
| | change | | | | | | |
| | within | | | | | | |
| | group | | | | | | |

TABLE 24
Changes in sPLA2 levels in statin subgroup of ITT population

| | | A-002 | A-002 | A-002 | A-002 | A-002 | |
|---|---|---|---|---|---|---|---|
| | | (50 mg) | (100 mg) | (250 mg) | (500 mg) | Total | Placebo |
| Baseline | # of | 15 | 14 | 14 | 15 | 58 | 14 |
| | subjects | | | | | | |
| | Mean (SD) | 3.34 ng/ml | 2.72 ng/ml | 2.84 ng/ml | 3.05 ng/ml | 3.00 ng/ml | 3.07 ng/ml |
| | | (2.08) | (1.53) | (1.40) | (1.37) | (1.60) | (1.69) |
| | Median | 2.70 ng/ml | 2.65 ng/ml | 2.40 ng/ml | 3.00 ng/ml | 2.70 ng/ml | 2.85 ng/ml |
| | Range | 0.80 to 8.80 | 0.05 to 6.00 | 1.30 to 5.20 | 0.60 to 5.40 | 0.05 to 8.80 | 0.80 to 5.70 |
| Week 2 | # of | 12 | 13 | 13 | 13 | 51 | 14 |
| | subjects | | | | | | |
| | Mean (SD) | 0.89 ng/ml | 0.66 ng/ml | 0.68 ng/ml | 0.20 ng/ml | 0.60 ng/ml | 3.08 ng/ml |
| | | (1.06) | (0.76) | (1.15) | (0.19) | (0.88) | (1.23) |
| | Median | 0.45 ng/ml | 0.40 ng/ml | 0.30 ng/ml | 0.10 ng/ml | 0.30 ng/ml | 3.10 ng/ml |
| | Range | 0.20 to 4.00 | 0.05 to 2.90 | 0.05 to 4.40 | 0.05 to 0.60 | 0.05 to 4.40 | 1.40 to 5.70 |
| Change from | # of | 12 | 13 | 12 | 13 | 50 | 14 |
| baseline at week 2 | subjects | | | | | | |
| | observed | | | | | | |
| | Mean (SD) | −2.74 ng/ml | −1.81 ng/ml | −2.01 ng/ml | −2.93 ng/ml | −2.37 ng/ml | +0.01 ng/ml |
| | | (1.60) | (1.68) | (1.26) | (1.32) | (1.51) | (1.41) |
| | Median | −2.60 ng/ml | −1.80 ng/ml | −1.65 ng/ml | −3.30 ng/ml | −2.43 ng/ml | +0.10 ng/ml |
| | Range | −5.30 to −0.60 | −3.70 to +2.85 | −4.20 to −0.20 | −4.80 to −0.55 | −5.30 to +2.85 | −1.80 to +3.90 |
| | p-value of | 0.0001 | 0.0105 | 0.0002 | <0.0001 | <0.0001 | 0.8003 |
| | change | | | | | | |
| | within | | | | | | |
| | group | | | | | | |
| Week 4 | # of | 13 | 12 | 13 | 13 | 51 | 13 |
| | subjects | | | | | | |
| | observed | | | | | | |
| | Mean (SD) | 0.85 ng/ml | 0.44 ng/ml | 0.57 ng/ml | 0.14 ng/ml | 0.50 ng/ml | 4.68 ng/ml |
| | | (0.61) | (0.34) | (0.65) | (0.17) | (0.54) | (3.82) |
| | Median | 0.70 ng/ml | 0.40 ng/ml | 0.30 ng/ml | 0.05 ng/ml | 0.30 ng/ml | 3.70 ng/ml |
| | Range | 0.10 to 2.40 | 0.05 to 1.00 | 0.05 to 2.30 | 0.05 to 0.60 | 0.05 to 2.40 | 1.30 to 15.90 |
| Change from | # of | 12 | 11 | 12 | 13 | 48 | 13 |
| baseline at week 4 | subjects | | | | | | |
| | observed | | | | | | |
| | Mean (SD) | −2.50 ng/ml | −2.46 ng/ml | −2.13 ng/ml | −2.99 ng/ml | −2.53 ng/ml | +1.45 ng/ml |
| | | (1.77) | (1.52) | (1.24) | (1.36) | (1.47) | (3.33) |
| | Median | −2.15 ng/ml | −2.50 ng/ml | −2.15 ng/ml | −3.50 ng/ml | −2.43 ng/ml | +0.40 ng/ml |
| | Range | −6.40 to −0.60 | −5.70 to 0.00 | −4.20 to +0.20 | −4.80 to −0.55 | −6.40 to +0.20 | −1.20 to |
| | | | | | | | +10.20 |
| | p-value of | 0.0005 | 0.0003 | <0.0001 | <0.0001 | <0.0001 | 0.4861 |
| | change | | | | | | |
| | within | | | | | | |
| | group | | | | | | |

TABLE 25
Changes in sPLA2 levels in non-statin subgroup of ITT population

| | | A-002 | A-002 | A-002 | A-002 | A-002 | |
|---|---|---|---|---|---|---|---|
| | | (50 mg) | (100 mg) | (250 mg) | (500 mg) | Total | Placebo |
| Baseline | # of | 1 | 2 | 1 | 2 | 6 | 2 |
| | subjects | | | | | | |
| | Mean (SD) | 1.80 ng/ml | 3.85 ng/ml | 3.40 ng/ml | 2.30 ng/ml | 2.92 ng/ml | 2.25 ng/ml |
| | | (—) | (1.48) | (—) | (0.71) | (1.16) | (0.07) |
| | Median | 1.80 ng/ml | 3.85 ng/ml | 3.40 ng/ml | 2.30 ng/ml | 2.80 ng/ml | 2.25 ng/ml |
| | Range | 1.80 to 1.80 | 2.80 to 4.90 | 3.40 to 3.40 | 1.80 to 2.80 | 1.80 to 4.90 | 2.20 to 2.30 |
| Week 2 | # of | 0 | 1 | 1 | 2 | 4 | 3 |
| | subjects | | | | | | |
| | Mean (SD) | — | 0.90 ng/ml | 1.30 ng/ml | 0.05 ng/ml | 0.58 ng/ml | 3.23 ng/ml |
| | | | (—) | (—) | (0.00) | (0.63) | (1.55) |
| | Median | — | 0.90 ng/ml | 1.30 ng/ml | 0.05 ng/ml | 0.48 ng/ml | 2.60 ng/ml |
| | Range | — | 0.90 to 0.90 | 1.30 to 1.30 | 0.05 to 0.05 | 0.05 to 1.30 | 2.10 to 5.00 |
| Change from baseline at | # of | 0 | 1 | 1 | 2 | 4 | 2 |
| week 2 | subjects | | | | | | |
| | observed | | | | | | |
| | Mean (SD) | — | −1.90 ng/ml | −2.10 ng/ml | −2.25 ng/ml | −2.13 ng/ml | +0.10 ng/ml |
| | | | (—) | (—) | (0.71) | (0.44) | (0.28) |
| | Median | — | −1.90 ng/ml | −2.10 ng/ml | −2.25 ng/ml | −2.00 ng/ml | +0.10 ng/ml |
| | Range | — | −1.90 to −1.90 | −2.10 to −2.10 | −2.75 to −1.75 | −2.75 to −1.75 | −0.10 to +0.30 |
| | p-value of | — | 1.000 | 1.000 | 0.1392 | 0.0024 | 0.7048 |
| | change | | | | | | |
| | within | | | | | | |
| | group | | | | | | |
| Week 4 | # of | 0 | 2 | 1 | 2 | 5 | 2 |
| | subjects | | | | | | |
| | observed | | | | | | |
| | Mean (SD) | — | 0.80 ng/ml | 1.20 ng/ml | 0.23 ng/ml | 0.65 ng/ml | 2.50 ng/ml |
| | | | (0.42) | (—) | (0.25) | (0.49) | (0.14) |
| | Median | — | 0.80 ng/ml | 1.20 ng/ml | 0.23 ng/ml | 0.50 ng/ml | 2.50 ng/ml |
| | Range | — | 0.50 to 1.10 | 1.20 to 1.20 | 0.05 to 0.40 | 0.05 to 1.20 | 2.40 to 2.60 |
| Change from baseline at | # of | 0 | 2 | 1 | 2 | 5 | 2 |
| week 4 | subjects | | | | | | |
| | observed | | | | | | |
| | Mean (SD) | — | −3.05 ng/ml | −2.20 ng/ml | −2.08 ng/ml | −2.49 ng/ml | +0.25 ng/ml |
| | | | (1.91) | (—) | (0.46) | (1.11) | (0.07) |
| | Median | — | −3.05 ng/ml | −2.20 ng/ml | −2.08 ng/ml | −2.20 ng/ml | +0.25 ng/ml |
| | Range | — | −4.40 to −1.70 | −2.20 to −2.20 | −2.40 to −1.75 | −4.40 to −1.70 | +0.20 to |
| | | | | | | | +0.30 |
| | p-value of | — | 0.2653 | 1.000 | 0.0989 | 0.0074 | 0.1257 |
| | change | | | | | | |
| | within | | | | | | |
| | group | | | | | | |

TABLE 26
Changes in sPLA2 levels in ITT subjects exhibiting at least 3 out of 5 metabolic syndrome criteria

| | | A-002 | A-002 | A-002 | A-002 | A-002 | |
|---|---|---|---|---|---|---|---|
| | | (50 mg) | (100 mg) | (250 mg) | (500 mg) | Total | Placebo |
| Baseline | # of | 9 | 5 | 3 | 8 | 25 | 5 |
| | subjects | | | | | | |
| | Mean (SD) | 3.47 ng/ml | 2.97 ng/ml | 2.90 ng/ml | 3.05 ng/ml | 3.17 ng/ml | 2.76 ng/ml |
| | | (2.42) | (2.31) | (1.57) | (1.18) | (1.87) | (1.67) |
| | Median | 2.70 ng/ml | 2.20 ng/ml | 2.20 ng/ml | 3.40 ng/ml | 2.70 ng/ml | 2.00 ng/ml |
| | Range | 0.80 to 8.80 | 0.05 to 6.00 | 1.80 to 4.70 | 1.20 to 4.40 | 0.05 to 8.80 | 1.10 to 5.20 |
| Week 2 | # of | 7 | 4 | 3 | 7 | 21 | 6 |
| | subjects | | | | | | |
| | Mean (SD) | 0.96 ng/ml | 1.20 ng/ml | 0.33 ng/ml | 0.16 ng/ml | 0.65 ng/ml | 2.73 ng/ml |
| | | (1.38) | (1.20) | (0.06) | (0.15) | (0.99) | (1.32) |
| | Median | 0.30 ng/ml | 0.80 ng/ml | 0.30 ng/ml | 0.05 ng/ml | 0.30 ng/ml | 2.40 ng/ml |
| | Range | 0.20 to 4.00 | 0.30 to 2.90 | 0.30 to 0.40 | 0.05 to 0.40 | 0.05 to 4.00 | 1.50 to 5.00 |
| Change from | # of | 7 | 4 | 2 | 7 | 20 | 5 |
| baseline at week 2 | subjects | | | | | | |
| | observed | | | | | | |
| | Mean (SD) | −2.89 ng/ml | −1.01 ng/ml | −1.65 ng/ml | −2.94 ng/ml | −2.41 ng/ml | −0.48 ng/ml |
| | | (1.64) | (2.67) | (0.35) | (1.17) | (1.75) | (1.20) |
| | Median | −2.50 ng/ml | −1.80 ng/ml | −1.65 ng/ml | −3.50 ng/ml | −2.45 ng/ml | −0.10 ng/ml |
| | Range | −5.30 to −0.60 | −3.30 to +2.85 | −1.90 to −1.40 | −4.00 to −1.15 | −5.30 to +2.85 | −1.80 to +0.80 |
| | p-value of | 0.0035 | 0.5034 | 0.0957 | 0.0006 | <0.0001 | 0.4227 |
| | change | | | | | | |
| | within | | | | | | |
| | group | | | | | | |
| Week 4 | # of | 8 | 4 | 3 | 7 | 22 | 4 |
| | subjects | | | | | | |
| | observed | | | | | | |
| | Mean (SD) | 0.81 ng/ml | 0.41 ng/ml | 0.27 ng/ml | 0.06 ng/ml | 0.43 ng/ml | 3.45 ng/ml |
| | | (0.73) | (0.41) | (0.15) | (0.02) | (0.55) | (1.47) |
| | Median | 0.65 ng/ml | 0.30 ng/ml | 0.30 ng/ml | 0.05 ng/ml | 0.25 ng/ml | 3.55 ng/ml |
| | Range | 0.10 to 2.40 | 0.05 to 1.00 | 0.10 to 0.40 | 0.05 to 0.10 | 0.05 to 2.40 | 1.90 to 4.80 |
| Change from | # of | 8 | 4 | 2 | 7 | 21 | 4 |
| baseline at week 4 | subjects | | | | | | |
| | observed | | | | | | |
| | Mean (SD) | −2.76 ng/ml | −2.78 ng/ml | −1.75 ng/ml | −3.04 ng/ml | −2.76 ng/ml | +0.27 ng/ml |
| | | (1.98) | (2.42) | (0.49) | (1.26) | (1.69) | (1.77) |
| | Median | −2.15 ng/ml | −2.70 ng/ml | −1.75 ng/ml | −3.70 ng/ml | −2.30 ng/ml | −0.25 ng/ml |
| | Range | −6.40 to −0.60 | −5.70 to 0.00 | −2.10 to −1.40 | −4.35 to −1.15 | −6.40 to 0.00 | −1.20 to +2.80 |
| | p-value of | 0.0056 | 0.1055 | 0.1257 | 0.0007 | <0.0001 | 0.7758 |
| | change | | | | | | |
| | within | | | | | | |
| | group | | | | | | |

1. The method of claim 9, wherein said one or more sPLA₂ inhibitors are selected from the group consisting of ((3-(2-Amino-1,2-dioxoethyl)-2-ethyl-1-(phenylmethyl)-1H-indol-4-yl)oxy)acetic acid, {9-[(phenyl)methyl]-5-carbamoylcarbazol-4-yl}oxyacetic acid, and pharmaceutically acceptable salts, solvates or prodrug derivatives thereof.

2. The method of claim 1, wherein said prodrug derivative is selected from the group consisting of [[3-(2-Amino-1,2-dioxoethyl)-2-ethyl-1-(phenylmethyl)-1H-indol-4-yl]oxy]acetic acid methyl ester and ((3-(2-amino-1,2-dioxoethyl)-2-methyl-1-(phenylmethyl)-1H-indol-4-yl)oxy)acetic acid N-morpholino ethyl ester.

3. The method of claim 1, wherein said subject has cardiovascular disease or one or more conditions associated with cardiovascular disease.

4. The method of claim 3, wherein said cardiovascular disease is selected from the group consisting of atherosclerosis, coronary artery disease (CAD), coronary heart disease (CHD), conditions associated with CAD and CHD, cervebrovascular disease and conditions associated with cerebrovascular disease, peripheral vascular disease and conditions associated with peripheral vascular disease, aneurysm, vasculitis, venous thrombosis, diabetes mellitus, and metabolic syndrome.

5. (canceled)

6. The method of claim 1 further comprising administering to said subject one or more statins.

7. The method of claim 6, wherein said one or more statins are selected from the group consisting of atorvastatin, cerivastatin, fluvastatin, lovastatin, mevastatin, pitavastatin, pravastatin, rosuvastatin, simvastatin, ezetimibe plus simvastatin, and pharmaceutically acceptable salts, solvates, stereoisomers, or prodrug derivatives thereof.

8. A method of decreasing sPLA₂ levels in a subject in need thereof comprising administering a therapeutically effective amount of one or more sPLA₂ inhibitors.

9. The method of claim 8, wherein said one or more sPLA₂ inhibitors are indole-based sPLA₂ inhibitors.

10. The method of claim 1, wherein said one or more sPLA₂ inhibitors are administed to said subject once a day.

11. A method of measuring sPLA₂ levels in a serum sample comprising:
a) obtaining one or more serum test samples from a subject;
b) diluting said one or more serum test samples with one or more diluents comprising 0.01% to 100% purified human serum to generate one or more diluted serum test samples;
c) applying said one or more diluted serum test samples to a plate pre-coated with a capture antibody that specifically binds sPLA₂;
d) applying an acetylcholinesterase (AChE) conjugate antibody to said plate, wherein said AChE conjugate antibody comprises AChE conjugated to an antibody that specifically binds sPLA₂ at a different epitope than the capture antibody;
e) incubating said plate at about 15 to 30° C.;
f) pouring out the contents of said plate and washing said plate one or more times;
g) adding Ellman's reagent to said plate and developing said plate at about 15 to 30° C.;
h) measuring the absorbance of said one or more diluted serum test samples at 400 to 420 nm; and
i) determining the concentration of sPLA₂ in said one or more diluted serum test samples using a standard curve that plots absorbance at 400 to 420 nm versus sPLA₂ concentration for one or more control samples of known sPLA₂ concentration, wherein said one or more control samples were diluted in the same manner as the one or more serum test samples.

12. The method of claim 11, wherein duplicate serum test samples are diluted with two or more different diluents containing different percentages of purified human serum.

13. The method of claim 11, wherein absorbance is measured at 405 nm.

14. The method of claim 11, wherein the incubation step recited in step (e) is carried out for about 15 to 25 minutes.

15. The method of claim 11, wherein the development step recited in step (g) is carried out for about 60 to 120 minutes.

16. The method of claim 11, wherein said AChE conjugate antibody comprises Fab′.

17. A kit for performing the method of claim 11.
